SEQUENCE LISTING (10 sequences)

SEQ ID NO: 1 - protein, 826 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 2 - protein, 169 aa, synthetic construct
ALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHAA

SEQ ID NO: 3 - protein, 668 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVAAPIQGSRNLLQGEELLRALDQVN

SEQ ID NO: 4 - protein, 1097 aa, synthetic construct
MDYKDDDDKSRGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYKLALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHNCMEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVAAPIQGSRNLLQGEELLRALDQVNEFEQKLISEEDL

SEQ ID NO: 5 - protein, 417 aa, synthetic construct
MDYKDDDDKSRGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYKLALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHNC

SEQ ID NO: 6 - protein, 1097 aa, synthetic construct
MDYKDDDDKSRGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYKLALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHAAMEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVAAPIQGSRNLLQGEELLRALDQVNEFEQKLISEEDL

SEQ ID NO: 7 - protein, 235 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 8 - protein, 1101 aa, synthetic construct
MDYKDDDDKSRGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDGALADANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYKLALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHNCMEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVAAPIQGSRNLLQGEELLRALDQVNEFEQKLISEEDL

SEQ ID NO: 9 - protein, 888 aa, synthetic construct
MDYKDDDDKSRKYPLLIGIGLSAVIGLLSCLIGYCSSKLALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHNCMEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVAAPIQGSRNLLQGEELLRALDQVNEFEQKLISEEDL

SEQ ID NO: 10 - protein, 892 aa, synthetic construct
MDYKDDDDKSRGALAKYPLLIGIGLSAVIGLLSCLIGYCSSKLALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHNCMEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVAAPIQGSRNLLQGEELLRALDQVNEFEQKLISEEDL

(19) United States
(12) Patent Application Publication
(10) Pub. No.: US 2024/0050523 A1
(43) Pub. Date: Feb. 15, 2024

(54) METHOD FOR INDUCING RESOLUTIVE MACROPHAGE AND USES THEROF

(71) Applicants: SHIFTBIO INC., Seoul (KR); KOREA INSTITUTE OF SCIENCE AND TECHNOLOGY, Seoul (KR)

(72) Inventors: Gi-Hoon NAM, Seoul (KR); Gi Beom KIM, Seoul (KR); Jae Hyun KIM, Seoul (KR); Yeji LEE, Seoul (KR); Jiyoung GOO, Seoul (KR); Seongeun CHO, Seoul (KR); Cherlhyun JEONG, Seoul (KR); In-San KIM, Seoul (KR)

(21) Appl. No.: 18/447,152

(22) Filed: Aug. 9, 2023

Related U.S. Application Data
(60) Provisional application No. 63/371,077, filed on Aug. 11, 2022.

(51) Int. Cl. A61K 38/17; A61K 9/127; A61K 47/65; A61P 1/16; A61P 29/00; A61K 47/69; A61K 47/64

(52) U.S. Cl. CPC A61K 38/1709; A61K 9/127; A61K 38/177; A61K 47/65; A61P 1/16; A61P 29/00; A61K 47/6911; A61K 47/64

(57) ABSTRACT

The present invention generally relates to an extracellular vesicle (EV) comprising a subunit of a heterodimeric transcription factor, an EV polypeptide, and a monomeric cis-cleaving intein and a method of using the EV.

### CROSS-REFERENCE TO RELATED APPLICATION

The application claims priority from Provisional Application No. 63/371,077 filed on Aug. 11, 2022, which is incorporated herein by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 8, 2023, is named “Seq_SIW-P30004.xml” and is 17,043 bytes in size. The sequence listing contained in this .XML file is part of the specification and is hereby incorporated by reference herein in its entirety.

### TECHNICAL FIELD

The present invention generally relates to an extracellular vesicle (EV) comprising a subunit of a heterodimeric transcription factor, an EV polypeptide, and a monomeric cis-cleaving intein and a method for inducing an resolutive macrophage by using the EV.

### BACKGROUND

Monocytes and macrophages are types of phagocytes, which are cells that protect the body by ingesting harmful foreign particles, bacteria, and dead or dying cells. In addition to monocytes and macrophages, phagocytes include neutrophils, dendritic cells, and mast cells.

Macrophages are classically known as large white blood cells that patrol the body and engulf and digest cellular debris, and foreign substances, such as pathogens, microbes, and cancer cells, through a process known as phagocytosis. In addition, macrophages, including tissue macrophages and circulating monocyte-derived macrophages, are important mediators of both the innate and adaptive immune system.

Macrophages are integral to maintaining balance in all tissues, acting as key responders to pathogens and environmental threats, as well as aiding in post-injury tissue recovery. Research from numerous groups over the last decade has revealed the extraordinary flexibility of macrophages, which undergo epigenetic programming in response to signals from their tissue environments. These signals can include endocrine or paracrine signals, cues from phagocytosed cells, microvesicles, and extracellular matrix molecules. Moreover, macrophages can engage directly with surface receptors on other resident cells within the tissue, immune cells recruited during an injury, and proteins in the extracellular space. Consequently, macrophages undertake diverse roles across development, acute responses to infections and tissue injuries, and tissue repair. Given the tissue and disease stage—specific functions of macrophages, therapies targeting them may potentially result in fewer off-target effects than less selective treatments. However, to achieve this, it will be necessary to carry out more precise molecular endotyping and targeting of specific macrophage subsets throughout the process of tissue injury and repair.

Meanwhile, a considerable amount of effort has been made to use EVs to deliver to desired target cells for therapeutic purposes a variety of therapeutic molecules, examples of which include protein reporters, enzymes, antibodies, intrabodies, single chain variable fragments, antibody fragments, affibodies, tumor suppressors, viral or bacterial inhibitors, cell component proteins, deoxyribonucleic acid (DNA) and/or ribonucleic acid (RNA) binding proteins, DNA repair inhibitors, nucleases, proteinases, integrases, transcription factors, growth factors, apoptosis inhibitors and inducers, toxins, structural proteins, neurotrophic factors, membrane transporters, nucleotide binding proteins, heat shock proteins, clustered regularly interspaced short palindromic repeats (CRISPR)-associated proteins, Cas9, cytokines, tumor necrosis factor superfamily (TNFSF) ligands, vaccine antigens, and any combination thereof. EVs that have therapeutic molecules loaded on inner surfaces of the EVs and scaffolds that can deliver therapeutic molecules to inner surfaces of EVs were proposed, as described in, for example, WO/2000/028001 and WO/2017/203260, which are incorporated herein by reference

### SUMMARY

An aspect of the present invention provides a method for inducing resolutive macrophage in a subject, the method comprising the step of administering to the subject in need thereof a therapeutically effective amount of a composition containing one or more extracellular vesicles (EVs) and one or more pharmaceutically acceptable carriers and/or excipients, wherein the EV or EVs comprise one or more polypeptide constructs comprising: (i) HIF-1α, a fragment of the HIF-1α, a variant of the HIF-1α, a fragment of the variant, or a variant of the fragment; (ii) one or more EV polypeptides; and (iii) one or more monomeric cis-cleaving inteins.

In some embodiments, the composition modifies polarization of macrophages to favor resolutive macrophages and/or modifies balance between the different subtypes of macrophages toward resolutive macrophages and/or induces differentiation of monocytes to resolutive macrophages and/or induces phenotype switching from macrophages to resolutive macrophages.

In some embodiments, the fragment of the HIF-1α defects oxygen-dependent degradation domain (ODD) from wild-type HIF-1α.

In some embodiments, the variant of the HIF-1α comprises the amino acid sequence of SEQ ID NO: 3.

In some embodiments, the EV polypeptide or polypeptides are selected from the group consisting of: (i) one or more polypeptides selected from the group consisting of CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD4OL, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), and PTGFRN; (ii) a fragment or fragments of the polypeptide or polypeptides; (iii) a variant or variants of the polypeptide or polypeptides; (iv) a variant or variants of the fragment or fragments; and (v) a fragment or fragments of the variant or variants.

In some embodiments, the monomeric cis-cleaving intein originates from the protein selected from the group consisting of Prp8, VMA1, DdRP, ThrRS, GLT, CHS, IF2 elF5B, DnaB, ClpP, RIR, Helicases, and MutS-like.

In some embodiments, the monomeric cis-cleaving intein comprises the amino acid sequence of SEQ ID NO: 2.

In some embodiments, the polypeptide construct or constructs further comprise one or more targeting moieties. The targeting moiety or moieties may target one or more monocytes and/or one or more monocyte-derived macrophages.

In some embodiments, the polypeptide construct or constructs further comprise one or more fusogenic peptides. the fusogenic peptide or peptides may be selected from the group consisting of (GALA)n (n is integer from 1 to 10), (KALA)n (n is integer from 1 to 10), INF7, influenza virus HA2, melittin, octa-arginine (R8) peptide, vesicular stomatitis virus glycoprotein (VSV-G), tat protein of HIV, HSV-1 gB, EBV gB, thgoto virus G protein, and AcMNPV gp64.

In some embodiments, the polypeptide construct or constructs comprise an amino acid sequence or sequences selected from the group consisting of SEQ ID Nos: 4, 8, 9 and 10.

In some embodiments, the subject is in status of (a) increased M1 and/or M1-like macrophages compared to normal condition and/or (b) inflammatory condition accompanying increased proportion and/or increased number of monocyte-derived macrophage compared to normal condition.

In some embodiments, the resolutive macrophage is selected from the group consisting of an Ly6C(low) macrophage and a MerTK-positive macrophage.

In some embodiments, the resolutive macrophage is selected from the group consisting of a pro-efferocytic macrophage and an anti-oxidant macrophage.A further aspect of the present invention provides a resolutive macrophage-inducing agent comprising an EV or EVs that comprise one or more polypeptide constructs, wherein the polypeptide construct or constructs comprise: (i) HIF-1α, a fragment of the HIF-1α, a variant of the HIF-1α, a fragment of the variant, or a variant of the fragment; (ii) an EV polypeptide; and (iii) a monomeric cis-cleaving intein.

A still further aspect of the present invention provides a method for preventing or treating inflammatory diseases, conditions, or symptoms, the method comprising administering to a subject in need thereof a prophylactically or therapeutically effective amount of a composition containing one or more EVs that comprise one or more polypeptide constructs, wherein the polypeptide construct or constructs comprise: (i) HIF-1α, a fragment of the HIF-1α, variant of the HIF-1α, a fragment of the variant, or a variant of the fragment; (ii) an EV polypeptide; and (iii) a monomeric cis-cleaving intein.

In some embodiments, the inflammatory disease, condition, or symptom is selected from the group consisting of single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, rheumatoid arthritis, glomerulonephritis, pancreatitis, hepatitis, non-alcoholic steatohepatitis(NASH), inflammatory arthritis, gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, crohn's and ulcerative colitis, terminal ileitis, alcoholic steatohepatitis, hepatotoxicity, liver infection, remote liver injury, lupus, autoimmune diseases associated with acute or chronic inflammation, and acute or chronic inflammation associated with viral, bacterial or fungal infection.

In some embodiments, the inflammatory disease, condition, or symptom is related to (a) increased M1 and/or M1-like macrophages compared to normal condition and/or (b) increased proportion and/or increased number of monocyte-derived macrophage compared to normal condition.

A still yet further aspect of the present invention provides an EV comprising one or more polypeptide construct or constructs that comprise (i) one or more fusogenic peptides, (ii) one or more polypeptides of interest (PoIs), (iii) one or more EV polypeptides, and (iv) one or more monomeric cis-cleaving inteins.

The above and other aspects and embodiments of the present invention will be discussed in more detail below.

### BRIEF DESCRIPTION OF THE DRAWINGS

FIG. 1 shows a schematic illustration of constructs of wild-type HIF1α and mutated HIF1α (scHIF 1α).

FIG. 2 presents a schematic illustration of a vector map containing CD81, intein, and scHIF1α, a vector map containing CD81, intein, and a stop codon, and a vector map containing CD81, mutated intein (Intein*, and scHIF1α.

FIG. 3 presents an illustration of a western blot analysis showing the cleavage activity of intein. CD81_Intein_scHIF1α (Full) displays a band below due to cleavage by intein, while CD81_Intein*_scHIF1α (Mut) does not undergo cleavage, resulting in the absence of the lower band. Lane 1 displays CD81_Intein_scHIF1α (Full) EV [scHIF1α-EV], Lane 2 shows CD81_Intein (Stop) EV [Ctrl-EV], and Lane 3 presents CD81_Intein*_scHIF1α (Mut) EV. Alix and tsg101 serve as EV positive markers, and calnexin is used as an EV negative marker.

FIG. 4 represents the outcomes of a western blot that confirms the enrichment of scHIF1α within EVs via the sorting capacity of CD81 and the cleavage activity of intein. mCherry is employed as a control for transfection efficiency. The graphs below show the intensity of each western blot band.

FIG. 5 displays the results of luciferase activity when EV-encapsulated scHIF1α [scHIF1α-EV] was delivered to HEK293 cells expressing luciferase under the VEGF promoter. scHIF1α-EV demonstrated an increased activity of VEGF compared to Ctrl-EV in this assay.

FIG. 6 depicts the results of a cytokine array analysis, which shows the expression of angiogenesis-related cytokines in macrophages sorted ex vivo from the liver using F4/80 magnetic beads and treated with EVs. Only the scHIF1α-EV demonstrated an increase in the release of angiogenesis-related cytokines.

FIG. 7 displays the qRT-PCR results of mRNA expression for VEGF and GLUT1, downstream genes of HIF1α, upon treatment with scHIF1α-EV. Only EV-encapsulated scHIF1α showed increased expression of VEGF and GLUT1.

FIG. 8 represents the results of evaluating vascular infiltration. A mixture of 50, 100 μg of scHIF1α-EVs and matrigel was subcutaneously injected into 7-week-old male NOD/SCID mice. Only scHIF1α-EV showed increased vascular infiltration in the matrigel.

FIG. 9 depicts the results of a 3-day survival rate test. LPS (15 mg/kg) was intraperitoneally injected into 7-week-old male C57BL/6 mice to induce a severe inflammatory disease condition. Subsequently, 50 μg of scHIF1α-EV was intravenously injected. Only the scHIF1α-EV group showed an increased survival rate over the 3-day period.

FIG. 10 illustrates the assessment of EV accumulation in major organs. Tissues were collected after intraperitoneal injection of LPS (5 mg/kg) and intravenous injection of 100 μg of Cy5-labeled EVs. Regardless of the groups, the liver exhibited the highest accumulation of EVs.

FIG. 11 illustrates that intravenous injection of 100 μg of Cy5-labeled EVs preferentially targeted monocytes and macrophages in the inflamed liver, which was induced by intraperitoneal injection of LPS (5 mg/kg).

FIG. 12 illustrates the results of measuring liver toxicity markers, ALT and AST, in the serum, as well as measuring pro-inflammatory cytokines, TNF-α and IL-6, through ELISA. Serums were collected after intraperitoneal injection of LPS (5 mg/kg) and intravenous injection of 30 μg of EVs. Significant decreases in all measured parameters were observed in the scHIF1α-EV group.

FIG. 13 shows the results of a cytokine array in the serum. Serums were collected after intraperitoneal injection of LPS (5 mg/kg) and intravenous injection of 30 μg of EVs. The scHIF1α-EV group demonstrated a decrease in overall pro-inflammatory cytokines and chemokines.

FIG. 14 presents the flow cytometry analysis results of immune cells in the damaged liver. The liver was collected after intraperitoneal injection of LPS (5 mg/kg) and intravenous injection of 30 μg of EVs. In the scHIF1α-EV group, there was an increase in the proportion of liver sinusoidal endothelial cells (LSEC) and a decrease in neutrophil infiltration.

FIG. 15 displays the flow cytometry analysis results of monocytes and monocytes derived macrophages (MoMFs) in the liver. The liver was collected after intraperitoneal injection of LPS (5 mg/kg) and intravenous injection of 30 μg of EVs. In the scHIF1α-EV group, there was an increased ratio of Ly6^{lo}/Ly6C^{hi} in MoMFs.

FIG. 16 presents the results of single cell RNA sequencing analysis performed on sorted CD45+ cells in the liver. The liver was collected after intraperitoneal injection of LPS (5 mg/kg) and intravenous injection of 30 μg of EVs. A total of 10 clusters were identified, and when treated with LPS, there was a significant increase in cluster 0 (activated monocyte/monocyte derived macrophages, activated Mo/MoMFs).

FIG. 17 is a schematic diagram that divides the significantly altered cluster 0, represented as activated Mo/MoMFs in FIG. 16, into 6 subclusters: pro-efferocytic macrophages (pro-efferocytic MFs, cluster 0), infiltrating CCR2+ macrophages (infiltrating CCR2+ MFs, cluster 1), anti-oxidant macrophages (anti-oxidant MFs, cluster 2), proliferating monocytes/macrophages (proliferating Mo/MFs, cluster 3), inflamed macrophages (inflamed MFs, cluster 4), and quiescent macrophages (quiescent MFs, cluster 5).

FIG. 18 is a heatmap depicting the collection of markers divided into 6 clusters derived from the Mo/MoMFs cluster, and the schematic diagrams below show the representative expression of markers for each cluster.

FIG. 19 is a graph depicting the proportions of 6 clusters derived from the Mo/MoMFs cluster for each group. Consistent with the flow cytometry results, a decrease in Ly6C2 and Itgam genes were observed in the scHIF1α-EV group.

FIG. 20 is a schematic diagram showing the differentiation direction of each cluster in Mo/MoMFs. In the scHIF1α-EV group, the expression of Cebpb, which is crucial for myeloid differentiation, increased, and the expression of Socs3, which inhibits cytokine receptor signaling, also increased.

FIG. 21 presents flow cytometry data analyzing kupffer cells obtained from tissues collected after intraperitoneal injection of LPS at 5 mg/kg and intravenous injection of EV at 30 μg. In the scHIF1α-EV group, the proportion of kupffer cells increased, and the proportion of MerTK-positive kupffer cells also increased.

FIG. 22-23 indicate that the expression of GALA on EVs can enhance the intracellular delivery rate of HIF1α into target cells. This increase was determined by functional tests such as the luciferase assay and CCK assay.

### DETAILED DESCRIPTION

### Definition

Unless defined otherwise, all technical and scientific terms used herein have the meaning commonly understood by a person skilled in the art to which this invention belongs. As used herein, the following terms have the meanings ascribed to them below.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, the term “a combination thereof” or “combinations thereof” refers to all permutations and combinations of the listed items preceding the term. For example, “A, B, C, or a combination thereof” or “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “about” is used to indicate that a value includes the inherent variation of error for the composition, the method used to administer the composition, or the variation that exists among the study subjects.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally, extracellular vesicles range in diameter from 20 nm to 1000 nm and can comprise various macromolecular cargo either within the internal space, displayed on the external surface of the extracellular vesicle, and/or spanning the membrane. The cargo can comprise small molecules, nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “exosome” refers to a cell-derived small vesicle comprising a membrane that encloses an internal space, and which is generated from said cell by direct plasma membrane budding or by fusion of the late endosome with the plasma membrane. The exosome comprises lipid or fatty acid and polypeptide and optionally comprises a therapeutic active payload, a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of EV.

As used herein, the terms “EV protein,” “EV polypeptide,” “exosomal protein,” “exosomal polypeptide,” and “exosome protein” are used interchangeably herein and shall be understood to relate to any protein or polypeptide that can be utilized to transport a polypeptide construct to an extracellular vesicle. More specifically, the term “EV protein” shall be understood as comprising any protein or polypeptide that enables transporting, trafficking or shuttling of a polypeptide construct to an EV, such as an exosome. Examples of such EV proteins are for instance CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD4OL, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), PTGFRN, a fragment thereof, a variant thereof, a variant of the fragment and a fragment of the variant and any combinations thereof, but numerous other polypeptides capable of transporting a polypeptide construct to EVs are comprised within the scope of the present invention. The EV proteins are typically of human origin and can be found in various publicly available databases such as Uniprot, RCSB, etc.

As used herein, the term “a fragment” of a protein, peptide, or nucleic acid refers to a segment of the protein, peptide, or nucleic acid. The fragments of the protein, peptide, or nucleic acid in accordance with some embodiments of the present invention may retain at least 30%, 35%, 40%, 45%, 50%, 55%, 60%, 65%, 70%, 75%, 80%, 85%, 90%, 95%, 99%, or 100% of the ability of the protein, peptide, or nucleic acid.

As used herein, the term “variant” of a protein, peptide, or nucleic acid refers to a protein, peptide, or nucleic acid having has at least one amino acid or nucleotide which is different from the protein, peptide, or nucleic acid. A variant of a protein, peptide, or nucleic acid includes, but is not limited to, a substitution, deletion, frameshift, or rearrangement in the protein, peptide, or nucleic acid. The term may be used interchangeably with the term “mutant.” The variants of the protein, peptide, or nucleic acid in accordance with some embodiments of the present invention may retain at least 30%, 35%, 40%, 45%, 50%, 55%, 60%, 65%, 70%, 75%, 80%, 85%, 90%, 95%, 99%, or 100% of the ability of the protein, peptide, or nucleic acid.

As used herein, the term “engineered EV” refers to an EV having therapeutic component or components at a desired position of the EV. In some embodiments, the desired position of the EV is an inner space of the EV.

As used herein, the term “fusogenic peptide” refers to a peptide that induces a homologous or target fusion between cells or membrane vesicles surrounded by a plasma membrane. The fusogenic peptide as such may include (GALA)n (n is an integer from 1 to 10), (KALA)n (n is an integer from 1 to 10), INF7, influenza virus HA2, melittin, octa-arginine (R8) peptide, vesicular stomatitis virus glycoprotein (VSV-G), tat protein of HIV, HSV-1 gB, EBV gB, thgoto virus G protein, AcMNPV gp64, a fragment thereof, a variant thereof, a variant of the fragment and a fragment of the variant and any combinations thereof, but numerous other peptides capable of transporting a polypeptide construct to EVs are comprised within the scope of the present invention. The integer is 1, 2, 3, 4, 5, 6, 7, 8, 9 or 10.

As used herein, the term “hypoxia-inducible factor 1” or “HIF-1” refers to a heterodimeric transcription factor that plays a critical role in regulating mammalian oxygen homeostasis. HIF-1 is a heterodimer of two subunits, HIF-1α and HIF-1β. The HIF-1α subunit is unique to HIF-1, whereas HIF-1β (also known as aryl hydrocarbon receptor nuclear translocator, ARNT) can dimerize with other proteins. HIF-1α-subunits are stabilized under hypoxic conditions and are important in regulating genes involved in angiogenesis and glucose metabolism. Structural analysis of HIF-1α revealed that dimerization requires two domains, termed HLH and PAS. DNA binding is mediated by a basic domain. Two transactivation domains are contained in HIF-1α, located between amino acids 531 and 826. The minimal transactivation domains are at amino acid residues 531-575 and 786-826. Amino acids 1-390 are required for optimal heterodimerization with HIF-1β and DNA binding. In addition, deletion of the carboxy terminus of HIF-1α (amino acids 391-826) decreased the ability of HIF-1 to activate transcription, as described in US6849718B2, which is incorporated herein by reference. However, HIF-1α (1-390) was expressed at elevated levels in both hypoxic and non-hypoxic cells in contrast to full-length HIF-1α (1-826) which was expressed at much higher levels in hypoxic relative to non-hypoxic cells. See, e.g., Semenza et al., Structural and functional analysis of hypoxia-inducible factor 1, Kidney Int, 1997 February;51(2):553-5; Jiang et al., Dimerization, DNA binding and transactivation properties of hypoxia-inducible factor-1. J Biol Chem, 1996 Jul. 26;271(30):17771-8, which are incorporated herein by reference. Under hypoxia, HIF-1 becomes activated and up-regulates target genes such as erythropoietin, vascular endothelial growth factor, glucose transporter, and glycolytic enzymes. HIF-1 activation is regulated primarily by the accumulation of HIF-1α protein. Both HIF-1α and HIF-1β genes are constitutively expressed in many cell lines, whereas HIF-1α protein is constantly degraded under normoxia by the ubiquitin-proteasome pathway. The degradation is controlled by a unique oxygen-dependent degradation domain (ODD) consisting of about 200 amino acids within HIF-1α. Deletion of the entire ODD gave rise to a stable HIF-1α, capable of heterodimerization, DNA-binding, and transactivation in cell culture systems. Consistently, the ODD-deleted HIF-1α in mammal can activate HIF-1 target genes irrespective of hypoxic signal.

As used herein, the term “intein” refers to internal protein fragments that disrupt the coding region of a host gene. These internal protein elements mediate the post-translational protein splicing process, catalyzing a series of reactions to remove the intein from the protein precursor and to ligate the flanking external protein fragments, known as exteins, into a mature protein. A typical intein element consists of 400 to 500 amino acid residues and contains four conserved protein splicing motifs (A, B, F, and G) which are separated by a homing endonuclease coding region. The endonuclease does not play a role in protein splicing and can be deleted from the intein sequence without impacting the inteins function. A few mini-inteins have been identified, which do not contain a homing endonuclease; these are approximately 150 amino acids in size. The majority of inteins mediate maturation of enzymes involved in replication, DNA repair, transcription, or translation. The intein further includes derivatives or modifications of known inteins including amino acid substitutions as long as these derivatives and modifications permit cleavage. The DNA encoding these inteins have been described and it is known that certain mutations of these intein sequences do not prevent an intein induced cleavage reaction. Naturally occurred inteins exist in several forms including full-length inteins, mini-inteins and naturally split inteins. The full-length inteins and mini-inteins are both cis-splicing inteins, with or without an endonuclease domain. Split inteins are trans-splicing inteins, with two fragments transcribed and translated by two independent genes. Examples of most common and well-known inteins in nuclear DNA (nDNA), chloroplast DNA (cpDNA) and eukaryotic virus (vDNA) genomes are listed in Table 1.

TABLE 1

| | Number of | |
|---|---|---|
| Protein | inteins | Distribution |
| nDNA | | |
| Prp8 | 104 | Pezizomycotina, Agaricomycotina, |
| | | Ustilaginomycotina, |
| | | Mucoromycota, Blastocladiomycota, |
| | | Chytridiomycota, Amoebozoa, |
| | | Choanoflagellida, Chlorophyta |
| VMA1 | 42 | Pezizomycotina, Saccharomycotina, |
| | | Taphrinomycotina, Puccinomycotina |
| DdRP | 21 | Pezizomycotina, Agaricomycotina, |
| | | Ustilaginomycotina, Bladtocladiomycota, |
| | | Chytridiomycota, Amoebozoa |
| ThrRS | 5 | Saccharomycotina |
| GLT | 5 | Pezizomycotina, Saccharomycotina |
| CHS | 4 | Pezizomycotina |
| IF2 eIF5B | 3 | Chytridiomycota, Glomeromycota |
| cpDNA | | |
| DnaB | 56 | Rhodophyta, Heterokonta, Cryptophyta |
| DdRP | 12 | Chlorophyta |
| ClpP | 3 | Chlorophyta |
| vDNA | | |
| DdDP | 67 | Phycodnaviridae, Iridoviridae, |
| | | Mimiviridae, unclassified dsDNA |
| | | viruses, unclassified viruses |
| RIR | 34 | Phycodnaviridae, Iridoviridae, |
| | | Mimiviridae, Marseilleviridae, |
| | | unclassified dsDNA viruses, |
| | | unclassified viruses |
| Helicases | 20 | Phycodnaviridae, Marseilleviridae, |
| | | unclassified dsDNA viruses, |
| | | unclassified viruses |
| DdRP | 9 | Phycodnaviridae, Mimiviridae, |
| | | unclassified dsDNA viruses |
| MutS-like | 2 | Mimiviridae |

As used herein, the term “monocyte” refers to myeloid-derived immune effector cells that circulate in the blood, bone marrow, and spleen and have limited proliferation in a steady state condition. Monocytes are found among peripheral blood mononuclear cells (PBMCs), which also comprise other hematopoietic and immune cells, such as B cells, T cells, NK cells, and the like. Monocytes are produced by the bone marrow from hematopoietic stem cell precursors called monoblasts. Monocytes have two main functions in the immune system: (1) they can exit the bloodstream to replenish resident macrophages and dendritic cells (DCs) under normal states, and (2) they can quickly migrate to sites of infection in the tissues and divide/differentiate into macrophages and inflammatory dendritic cells to elicit an immune response in response to inflammation signals. Monocytes are usually identified in stained smears by their large bilobate nucleus. Monocytes also express chemokine receptors and pathogen recognition receptors that mediate migration from blood to tissues during infection. They produce inflammatory cytokines and phagocytose cells. In some embodiments, monocytes and/or macrophages of interest are identified according to CD11b+ expression and/or CD14+ expression.

As used herein, the term “macrophages” refers to critical immune effectors and regulators of inflammation and the innate immune response. Macrophages are heterogeneous, tissue-resident, terminally-differentiated, innate myeloid cells, which have remarkable plasticity and can change their physiology in response to local cues from the microenvironment and can assume a spectrum of functional requirements from host defense to tissue homeostasis. Macrophages are present in virtually all tissues in the body. They are either tissue resident macrophages, for example Kupffer cells that reside in liver, or derived from circulating monocytic precursors (i.e., monocytes) which mainly originate from bone marrow and spleen reservoirs and migrate into tissue in the steady state or in response to inflammation or other stimulating cues. For example, monocytes can be recruited from the blood to tissue to replenish tissue specific macrophages of the bone, alveoli (lung), central nervous system, connective tissues, gastrointestinal tract, liver, spleen, and peritoneum. The macrophages include tissue-resident macrophages and circulating monocyte-derived macrophages.

As used herein, the term “tissue-resident macrophages” refers to heterogeneous populations of immune cells that fulfill tissue-specific and/or micro-anatomical niche-specific functions such as tissue immune-surveillance, response to infection and the resolution of inflammation, and dedicated homeostatic functions. Tissue resident macrophages originate in the yolk sac of the embryo and mature in one particular tissue in the developing fetus, where they acquire tissue-specific roles and change their gene expression profile. Local proliferation of tissue resident macrophages, which maintain colony-forming capacity, can directly give rise to populations of mature macrophages in the tissue. Tissue resident macrophages can also be identified and named according to the tissues they occupy.

As used herein, the term “Ml macrophages” or “classically activated macrophages” refers to macrophages having a pro-inflammatory phenotype. The term “macrophage activation” (also referred to as “classical activation”) was introduced by Mackaness in the 1960s in an infection context to describe the antigen-dependent, but non-specific enhanced, microbicidal activity of macrophages toward BCG (bacillus Calmette-Guerin) and Listeria upon secondary exposure to the pathogens (Mackaness (1962). Exp. Med. 116:381-406, which is incorporated herein by reference.). The enhancement was later linked with Th1 responses and IFN-γ production by antigen-activated immune cells and extended to cytotoxic and antitumoral properties. Therefore, any macrophage functionality that enhances inflammation by cytokine secretion, antigen presentation, phagocytosis, cell-cell interactions, migration, etc. is considered pro-inflammatory, vitro and in vivo assays can measure different endpoints: general in vitro measurements include pro-inflammatory cell stimulation as measured by proliferation, migration, pro-inflammatory Th1 cytokine/chemokine secretion and/or migration, while general in vivo measurements further include analyzing pathogen fighting, tissue injury immediate responders, other cell activators, migration inducers, etc. For both in vitro and in vivo, pro-inflammatory antigen presentation can be assessed. Bacterial moieties, such as lipopolysaccharide (LPS), certain Toll-like receptor (TLR) agonists, the Th1 cytokine interferon-gamma (IFNy) (e.g., IFNγ produced by NK cells in response to stress and infections, and T helper cells with sustained production, which is incorporated herein by reference.) and TNF polarize macrophages along the M1 pathway. Activated M1 macrophages phagocytose and destroy microbes, eliminate damaged cells (e.g., tumor cells and apoptotic cells), present antigen to T cells for increasing adaptive immune responses, and produce high levels of pro-inflammatory cytokines (e.g., IL-1, IL-6, and IL-23), reactive oxygen species (ROS), and nitric oxide (NO), as well as activate other immune and non-immune cells. Characterized by their expression of inducible nitric oxide synthase (iNOS), reactive oxygen species (ROS), and production of the Thl-associated cytokine, IL-12, M1 macrophages are well-adapted to promote a strong immune response.

As used herein, the term “Type 1” or “M1-like” monocyte and/or macrophage is a monocyte and/or macrophage capable of contributing to a pro-inflammatory response that is characterized by at least one of the following: producing inflammatory stimuli by secreting at least one pro-inflammatory cytokine, expressing at least one cell surface activating molecule/a ligand for an activating molecule on its surface, recruiting/instructing/interacting with at least one other cell (including other macrophages and/or T cells) to stimulate pro-inflammatory responses, presenting antigen in a pro-inflammatory context, migrating to the site allowing for pro-inflammatory response initiation or starting to express at least one gene that is expected to lead to pro-inflammatory functionality. In certain embodiments, such pro-inflammatory state can be measured in a number of well-known manners, including, without limitation, one or more of a) increased cluster of differentiation 80 (CD80), CD86, MHCII, MHCI, interleukin 1-beta (IL-1β, IL-6, CCL3, CCL4, CXCL10, CXCL9, GM-CSF and/or tumor necrosis factor alpha (TNF-α); b) decreased expression and/or secretion of CD206, CD163, CD16, CD53, VSIG4, PSGL-1, TGFb and/or IL-10; c) increased secretion of at least one cytokine or chemokine selected from the group consisting of IL-1β, TNF-α, IL-12, IL-18, GM-CSF, CCL3, CCL4, and IL-23; d) increased ratio of expression of IL-1β00, IL-6, and/or TNF-a to expression of IL-10; e) increased CD8+ cytotoxic T cell activation; f) increased recruitment of CD8+ cytotoxic T cell activation; g) increased CD4+ helper T cell activity; h) increased recruitment of CD4+ helper T cell activity; i) increased NK cell activity; j) increased recruitment of NK cell; k) increased neutrophil activity; l) increased macrophage activity; and/or m) increased spindle-shaped morphology, flatness of appearance, and/or number of dendrites, as assessed by microscopy.

As used herein, the term “resolutive macrophages” refers to macrophages contributing to resolution of inflammation and tissue repair, and can be interchangeably used with “pro-resolving macrophages”, “resolving macrophages”, “M2-like macrophages”, “proresolution macrophage”, or “resolution-phase macrophage”. Macrophages play critical roles in tissue remodeling in normal physiology and in resolution of inflammation and tissue injury. A critical process in resolution is the clearance of apoptotic cells (Acs), or efferocytosis. Efferocytosis prevents tissue necrosis and inflammation by preventing necrosis of dead cells, and apoptotic cells activate receptor-mediated signaling pathways in macrophages that suppress inflammation and promote resolution and repairment of inflammatory tissues. Macrophages involved in such process (i.e., suppression of inflammation, promotion of resolution and repairment of inflammatory tissues and legions) and capable of high-capacity efferocytosis may be defined as resolutive macrophages. Historically, attempts have been made to interpret biological phenomena by simply segregating macrophages into M1 and M2 categories. The M1/M2 polarization framework served as a valuable asset for in vitro macrophage research. Nevertheless, its utilization has convoluted our understanding of macrophage plasticity in vivo, considering that conventional M1 and M2 polarization is less likely to occur in tissue-specific contexts. Amid acute injury, the M1/M2 paradigm can provoke misinterpretations, as tissue-resident and monocyte-derived macrophages cohabitate within the wounded microenvironment. To circumvent these constraining factors, researchers now harness sophisticated methodologies such as RNA sequencing and flow cytometry to facilitate a more intricate and holistic classification of macrophage phenotypes. In some embodiments, the resolutive macrophages may comprise Ly6C(low) macrophages and MerTK-positive (i.e., MerTK+) macrophages. In some embodiments, the resolutive macrophages may comprise pro-efferocytic macrophages and anti-oxidant macrophages. In some embodiments, the pro-efferocytic macrophages are macrophages expressing at least one marker selected from the group consisting of Ly6c2^{low}, MerTK, Marco, Stab1, Arg1, Nr1h3, id1/3, Hmox1, il1rn, Ccl6, Ccl12, Mmp14. In some embodiments, the pro-efferocytic macrophage are macrophages expressing at least two, three, four, five, six, seven, eight, nine, ten, eleven or twelve markers selected from the group consisting of Ly6c2^{low}, MerTK, Marco, Stab1, Arg1, Nr1h3, id1/3, Hmox1, il1rn, Ccl6, Ccl12 and Mmp14. In some embodiments, the pro-efferocytic macrophages are Ly6c2^{low}, MerTK+, Arg1+, Nr1h3+, id1/3+, Hmox1+ and Mmp14+ macrophages. In a preferred embodiment, the pro-efferocytic macrophages are Ly6c2^{low}, MerTK+, Marco+, Stab1+, Arg1+, Nr1h3+, id1/3+, Hmox1+, il1rn+, Ccl6+, Ccl12+and Mmp14+ macrophages. In some embodiments, the anti-oxidant macrophages are macrophages expressing at least one marker selected from the group consisting of Ly6c2^{low}, C/EBPβ, Nr4a1, Pparg, Stat3, Nfe212, Bcl2, Csf1r, il1b, il17ra, Pecam1 and ifngr1. In some embodiments, the anti-oxidant macrophages are macrophages expressing at least two, three, four, five, six, seven, eight, nine, ten, eleven or twelve markers selected from the group consisting of Ly6c2^{low}, C/EBPβ, Nr4a1, Pparg, Stat3, Nfe212, Bcl2, Csf1r, il1b, il17ra, Pecam1, ifngr1. In some embodiments, the anti-oxidant macrophages are Ly6c2^{low}, C/EBPβ+, Nr4a1+, Pparg+, Nfe212+ and Csf1r+ macrophages. In a preferred embodiment, the anti-oxidant macrophages are Ly6c2^{low}, C/EBPβ+, Nr4a1+, Pparg+, Stat3+, Nfe212+, Bcl2+, Csf1r+, il1b+, ilb 17ra+, Pecam 1+ and ifngr1+ macrophages. see, e.g., Brennan et al., Efferocytosis induces macrophage proliferation to help resolve tissue injury, Cell Metab, 2021 December;33(12):2445-2463; Amanda et al., Efferocytosis in health and disease, Nat Rev Immunol, 2020 April;20(4):254-267; Jonas et al., Resolution-phase macrophages possess a unique inflammatory phenotype that is controlled by cAMP, Blood, 2008 November;112(10):4117-27; Satoshi et al., The role of macrophages in the resolution of inflammation, J Clin Invest. 2019 May;129(7):2619-2628; Johnathan et al., Scavenger receptors in homeostasis and immunity, Nat Rev Immunol, 2013 September;13(9):621-34; Giulia et al., Macrophage subsets in atherosclerosis, Nat Rev Cardiol. 2015 January;12(1):10-7, which are incorporated herein by reference.

As used herein, the term “activated” or “activation” refers to the state of a monocyte and/or macrophage that has been sufficiently stimulated to induce detectable cellular proliferation and/or has been stimulated to exert its effector function, such as induced cytokine expression and secretion, phagocytosis, cell signaling, antigen processing and presentation, target cell killing, and pro/anti-inflammatory function.

As used herein, the term “polarization” refers to the phenotypic features and the functional features of the macrophages. The phenotype can be defined through the markers expressed by the macrophages. The functionality can be defined for example based on the nature and the quantity of chemokines and/or cytokines expressed, in particular secreted, by the macrophages. Indeed, the macrophages present different phenotypic and functional features depending on their state.

As used herein, the term “resolutive macrophages inducing” refers to modifying polarization of macrophages to favor resolutive macrophages and/or modifying balance between the different subtypes of macrophages toward resolutive macrophages and/or inducing differentiation of monocytes to resolutive macrophages and/or inducing phenotype switching from macrophages to resolutive macrophages.

As used herein, the term “producer cell” refers to a cell used for generating an EV or an engineered EV. A producer cell includes, but is not limited to, a cell known to be effective in generating EVs, e.g., HEK293 cells, Chinese hamster ovary (CHO) cells, and mesenchymal stem cells (MSCs). The producer cell may be transformed or transfected by one or more vectors that contain or contains exogenous sequence or sequences. In some embodiments of the present invention, the producer cell can be transformed or transfected by one single vector that contains an exogenous sequence encoding the polypeptide construct. In some embodiments, the producer cell can be transformed or transfected by one single vector that contains an exogenous sequence encoding the polypeptide construct and an exogenous sequence encoding a therapeutically active payload. In some embodiments, the producer cell can be transformed or transfected by a vector that contains an exogenous sequence encoding the polypeptide construct and another vector that contains an exogenous sequence encoding a therapeutically active payload. In some embodiments, the producer cell can be transformed or transfected with at least one additional exogeneous sequence encoding another protein or peptide (e.g., a target moiety). The additional exogenous sequence can be introduced into the vector that contains an exogenous sequence encoding the polypeptide construct, an exogenous sequence encoding a therapeutically active payload, or both. In some embodiments, the exogenous sequence encoding a therapeutically active payload, the additional exogeneous sequence encoding another protein or peptide, or both can be introduced into the producer cell so as to modulate endogenous gene expression of the producer cell. In some embodiments, the exogenous sequence encoding a therapeutically active payload, the additional exogeneous sequence encoding another protein or peptide, or both can be introduced into the producer cell so as to produce the engineered EV that contains the therapeutically active payload, another protein or peptide, or both on the surface of the EV. The genetic construct (or vector) can be introduced into the EV-producing producer cells by any conventional method, such as by naked DNA technique, cationic lipid-mediated transfection, polymer-mediated transfection, peptide-mediated transfection, virus-mediated infection, physical or chemical agents or treatments, electroporation, etc. The EVs produced by such producer cells may be collected and/or purified according to techniques known in the art, such as by centrifugation, chromatography, etc., as described in WO00/44389 and U.S. Ser. No. 09/780,748, which are incorporated therein by reference.

As used herein, the term “vector” refers to carrier DNA molecules or DNA construct for introducing a desired gene into host cells and amplifying and expressing the desired gene. Preferably, vectors have auxotrophic genes, and have known restriction sites and the ability to replicate in hosts. In general, vectors may comprise a promoter, an enhancer, a terminator, SD sequence, translation initiation and termination codons, and a replication origin. If required, vectors may further comprise selection markers for selecting cells to which the vectors have been introduced. Such selection markers include: genes resistant to drugs such as ampicillin, tetracycline, kanamycin, chloramphenicol, neomycin, hygromycin, puromycin, and zeocin; markers that allow the selection using as an indicator an activity of an enzyme such as galactosidase; and markers such as GFP that allow selection using fluorescence emission as an indicator. It is also possible to use selection markers that allow selection using as an indicator a surface antigen such as EGF receptor and B7-2. By using such selection markers, only cells into which vectors have been introduced, more specifically cells into which the vectors of the present invention have been introduced, can be selected. The vectors may comprise signal sequences for polypeptide secretion. There is no limitation on the type of vectors to be used in the present invention; any vector may be used. In some embodiments, the vector is selected from the group consisting of a pET-vector, a pBAD-vector, a pK184-vector, a pMONO-vector, a pSELECT-vector, pSELECT-Tag-vector, a pVITRO-vector, a pVIVO-vector, a pORF-vector, a pBLAST-vector, a pUNO-vector, a pDUO-vector, a pZERO-vector, a pDeNy-vector, a pDRIVE-vector, a pDRIVE-SEAP-vector, a HaloTag®Fusion-vector, a pTARGET™-vector, a Flexi®-vector, a pDEST-vector, a pHIL-vector, a pPIC-vector, a pMET-vector, a pPink-vector, a pLP-vector, a pTOPO-vector, a pBud-vector, a pCEP-vector, a pCMV-vector, a pDisplay-vector, a pEF-vector, a pFL-vector, a pFRT-vector, a pFastBac-vector, a pGAPZ-vector, a pIZ/V5-vector, a pLenti6-vector, a pMIB-vector, a pOG-vector, a pOpti-vector, a pREP4-vector, a pRSET-vector, a pSCREEN-vector, a pSecTag-vector, a pTEF1-vector, a pTracer-vector, a pTrc-vector, a pUB6-vector, a pVAX1-vector, a pYC2-vector, a pYES2-vector, a pZeo-vector, a pcDNA-vector, a pFLAG-vector, a pTAC-vector, a pT7-vector, a Gateway®-vector, a pQE-vector, a pLEXY-vector, a pRNA-vector, a pPK-vector, a pUMVC-vector, a pLIVE-vector, a pCRUZ-vector, a Duet-vector, and other vectors or derivatives thereof.

As used herein, the term “therapeutically active payload” refers to a therapeutic agent capable of acting on a target (e.g., monocytes and/or macrophages) that is contacted with an EV. In some embodiments, the therapeutically active payload can be introduced into an EV. In some embodiments, the therapeutically active payload can be introduced into a producer cell. Non-limiting examples of the therapeutically active payload include nucleotides, nucleic acids (e.g., DNA mRNA, miRNA, dsDNA, lncRNA, and siRNA), amino acids, polypeptides, lipids, carbohydrates, and small molecules.

As used herein, the term “linker” refers to any molecular structure that can conjugate a peptide or a protein to another molecule (e.g., a different peptide or protein, a small molecule, etc.). Suitable linkers are well known to those of skill in the art and include, but are not limited to, straight or branched-chain carbon linkers, heterocyclic carbon linkers, or peptide linkers (see, e.g., Chen et al., Advanced Drug Delivery Reviews, 2013, Vol. 65:10, pp. 1357-1369, which is incorporated herein by reference.). The linkers can be joined to the carboxyl and amino terminal amino acids through their terminal carboxyl or amino groups or through their reactive side-chain groups. In addition, in some embodiments, linkers can be classified as flexible or rigid, and they can be cleavable (e.g., comprise one or more protease-cleavable sites, which can be located within the sequence of the linker or flanking the linker at either end of the linker sequence).

As used herein, the term “pharmaceutically acceptable” refers to compounds and compositions which are suitable for administration to humans and/or animals without undue adverse side effects such as toxicity, irritation and/or allergic response commensurate with a reasonable benefit/risk ratio.

As used herein, the term “biologically active” refers to the ability to modify the physiological system of an organism without reference to how the active agent has its physiological effects.

As used herein, the terms “subject” and “patient” are used interchangeably herein and will be understood to encompass mammals and non-mammals. Examples of mammals include, but are not limited to, humans, chimpanzees, apes monkeys, cattle, horses, sheep, goats, swine, rabbits, dogs, cats, rats, mice, guinea pigs, and the like. Examples of non-mammals include, but are not limited to, birds, fishes, and the like.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “administration” or “administering” of a composition refers to providing a composition to a subject in need of treatment. In accordance with embodiments of the present invention, therapeutic compositions may be administered singly or in combination with one or more additional therapeutic agents. The methods of administration of such compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration.

As used herein, the terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art. The compositions of the present disclosure may be administered by way of known pharmaceutical formulations, including tablets, pills, capsules, a liquid, an inhalant, a nasal spray solution, a suppository, a solution, a gel, an emulsion, an ointment, eye drops, ear drops, and the like.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising an engineered EV as disclosed herein required to provide a clinically significant decrease in disease symptoms. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skills in the art using routine experimentation based on the information provided herein.

As used herein, the term “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower disease burden, the prophylactically effective amount in some aspects will be higher than the therapeutically effective amount.

As used herein, the terms “polypeptide of interest”, “protein of interest”, “therapeutic polypeptide of interest”, “PoI”, “biotherapeutic”, “biologic”, and “protein biologic” are used interchangeably herein and shall be understood to relate to any polypeptide that can be utilized for therapeutic purposes through e.g. binding a target and/or in any other way interacting with an interaction partner and/or replace a protein and/or supplement or complement an existing intracellular protein, thereby exerting its therapeutic effect. Said terms may represent the following non-limiting examples of therapeutic polypeptides of interest: antibodies, intrabodies, single chain variable fragments (scFv), affibodies, bi-och multispecific antibodies or binders, receptors, ligands, enzymes for e.g. enzyme replacement therapy or gene editing, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. In one preferred embodiment, the PoI is a CRISPR-associated (Cas) polypeptide with intact nuclease activity which is associated with (i.e., carries with it) an RNA strand that enables the Cas polypeptide to carry out its nuclease activity in a target cell once delivered by the EV. Alternatively, in another preferred embodiment, the Cas polypeptide may be catalytically inactive, to enable targeted genetic engineering. Yet another alternative may be any other type of CRISPR effector such as the single RNA-guided endonuclease Cpf1. The inclusion of Cpf1 as the PoI is a particular preferred embodiment of the present invention, as it cleaves target DNA via a staggered double-stranded break, Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression. Additional preferred embodiments include PoIs selected from the group comprising enzymes for lysosomal storage disorders, for instance glucocerebrosidases such as imiglucerase, alpha-galactosidase, alpha-L-iduronidase, iduronate-2-sulfatase and idursulfase, aryl sulfatase, gal sulfase, acid-alpha glucosidase, sphingomyelinase, galactocerebrosidase, gal actosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alpha-N-acetyl neuraminidase, GlcNAc phosphotransferase, mucolipinl, palmitoyl-protein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase. In other preferred embodiments, the PoI may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The PoIs are typically proteins or peptides of human origin unless indicated otherwise by their name, any other nomenclature, or as known to a person skilled in the art, and they can be found in various publicly available databases such as Uniprot, RCSB, etc.

As used herein, the term “released inside” as in the context of “released inside an EV” or “released inside a target cell” can be understood to mean release of a polypeptide of interest (PoI) completely and/or partially inside the EV (or the target cell), i.e. that a PoI is released into the lumen of an EV (or the target cell), into the membrane of an EV (or the target cell) either completely or partially (e.g. into a transmembrane configuration) or onto the outside of the EV (or the target cell) membrane. Said term may also be understood to mean release of a PoI onto the external side of the EV membrane (or the target cell). Furthermore, it may also include being released inside any biological system, e.g., a particular tissue or a target organ.

As used herein, the terms “endogenous activation”, “endogenous triggering” and variants thereof (such as “endogenously activatable” or “endogenously triggered”) shall be understood to relate to activation, induction, and/or triggering of release of the PoI by the release system without any exogenous stimuli, i.e. the release of the PoI is triggered inside an EV or inside a cell by the mere action of the surrounding exosomal and/or cellular and/or biological environment (e.g. as a result of changes in pH, changes in other physiological parameters such as salinity, competition between binding partners, enzymatic activity e.g. proteolytic activity, etc.).

### EMBODIMENTS

In an aspect, the present invention provides a method for inducing a resolutive macrophage in a subject, the method comprising the step of administering to the subject in need thereof a therapeutically effective amount of extracellular vesicle (EV) comprising at least one polypeptide construct comprising: (i) HIF-1α, a fragment of HIF-1α, a variant of HIF-1α, a fragment of the variant, or a variant of the fragment; (ii) at least one EV polypeptide; and (iii) a monomeric cis-cleaving intein.

In some embodiments, the EV comprising at least one polypeptide construct may modify polarization of macrophages to favor resolutive macrophages and/or modify balance between the different subtypes of macrophages toward resolutive macrophages and/or inducing differentiation of monocytes to resolutive macrophages and/or inducing phenotype switching from macrophages to resolutive macrophages in a sample and/or a subject as compared to at least one reference sample and/or subject (e.g., a sample taken from the same subject prior to the treatment of the agent or historical data). For example, the EV comprising at least one polypeptide construct may modify polarization of macrophages to favor resolutive macrophages and/or modify balance between the different subtypes of macrophages toward resolutive macrophages and/or inducing differentiation of monocytes to resolutive macrophages and/or inducing phenotype switching from macrophages to resolutive macrophages in a sample and/or a subject with inflammatory condition (e.g., a sample or a subject treated with LPS, a patient having inflammatory diseases) as compared to at least one reference sample and/or subject (e.g., a sample taken from the same subject prior to the treatment of the agent or historical data).

In some embodiments, the EV comprising at least one polypeptide construct may increase the percentage of the population of resolutive macrophages in a sample and/or a subject as compared to at least one reference sample and/or subject (e.g., a sample taken from the same subject prior to the treatment of the agent or historical data). For example, the EV comprising at least one polypeptide construct may increase the percentage of the population of resolutive macrophages in a sample and/or a subject with inflammatory condition (e.g., a sample or a subject treated with LPS, a patient having inflammatory diseases) as compared to at least one reference sample and/or subject (e.g., a sample taken from the same subject prior to the treatment of the agent or historical data).

In some embodiments, the EV comprising at least one polypeptide construct may increase the percentage of the population of pro-efferocytic macrophages and/or anti-oxidant macrophages in a sample and/or a subject as compared to at least one reference sample and/or subject (e.g., a sample taken from the same subject prior to the treatment of the agent or historical data). For example, the EV comprising at least one polypeptide construct may increase the percentage of the population of pro-efferocytic macrophages and/or anti-oxidant macrophages in a sample and/or a subject with inflammatory condition (e.g., a sample or a subject treated with LPS, a patient having inflammatory diseases) as compared to at least one reference sample and/or subject (e.g., a sample taken from the same subject prior to the treatment of the agent or historical data).

In some embodiments, the EV comprising at least one polypeptide construct may increase the percentage of the population of Ly6C(low) macrophages in a sample and/or a subject as compared to at least one reference sample and/or subject (e.g., a sample taken from the same subject prior to the treatment of the agent or historical data). For example, the EV comprising at least one polypeptide construct may increase the percentage of the population of Ly6C(low) macrophages in a sample and/or a subject with inflammatory condition (e.g., a sample or a subject treated with LPS, a patient having inflammatory diseases) as compared to at least one reference sample and/or subject (e.g., a sample taken from the same subject prior to the treatment of the agent or historical data).

In some embodiments, the EV comprising at least one polypeptide construct may increase the percentage of the population of MerTK-positive macrophages in a sample and/or a subject as compared to at least one reference sample and/or subject (e.g., a sample taken from the same subject prior to the treatment of the agent or historical data). For example, the EV comprising at least one polypeptide construct may increase the percentage of the population of MerTK-positive macrophages in a sample and/or a subject with inflammatory condition (e.g., a sample or a subject treated with LPS, a patient having inflammatory diseases) as compared to at least one reference sample and/or subject (e.g., a sample taken from the same subject prior to the treatment of the agent or historical data).

In some embodiments, the EV comprising at least one polypeptide construct may induce resolutive macrophages in vitro, ex vivo and in vivo.

In some embodiments, the EVs may be surface-engineered EVs to comprise the polypeptide construct, in order to enhance their activity. The term “surface-engineered EV” refers to an EV whose membrane composition is modified. For example, the surface-engineered EVs may have a polypeptide construct on the surface of the EVs at a higher (or lower) density than a naturally occurring EVs do. In the present invention, a surface-engineered EVs can be produced from a genetically-engineered producer cell or a progeny thereof. For example, a surface-engineered EVs can be produced from stem cells transformed or transfected with an exogenous sequence or a DNA construct encoding the polypeptide construct.

In some embodiments, the HIF-1α may be a naturally-occurring (i.e., wild-type) HIF-1α. The wild-type HIF-1α comprises a protein having the same amino acid sequence as HIF-1α derived from nature, regardless of its mode of preparation. Thus, wild-type HIF-1α can have the amino acid sequence of naturally-occurring human HIF-1α, murine HIF-1α, or HIF-1α from any other mammalian species. For example, full-length wild-type sequence human HIF-1α is as shown in, e.g., NCBI reference sequence NOs: NP_001230013, NP_001521, NP_851397, NP_001521.1, NP_001300848, NP_001300849 or NP_034561, but not limited thereto. The wild-type HIF-1α may comprise naturally occurring prepro, pro and mature forms and truncated forms of HIF-1α, naturally-occurring variant forms (e.g., alternatively spliced forms), and naturally-occurring allelic variants. An exemplary sequence for the wild-type full-length HIF-1α consist of SEQ ID NO: 1;

SEQ ID NO: 1
MEGAGGANDKKKISSERRKEKSRDAARSRRSKESEVFYELAHQLPLPHNVSSHLDKASVMRL
TISYLRVRKLLDAGDLDIEDDMKAQMNCFYLKALDGFVMVLTDDGDMIYISDNVNKYMGLTQ
FELTGHSVEDFTHPCDHEEMREMLTHRNGLVKKGKEQNTQRSFFLRMKCTLTSRGRIMNIKS
ATWKVLHCTGHIHVYDTNSNQPQCGYKKPPMTCLVLICEPIPHPSNIEIPLDSKTFLSRHSL
DMKFSYCDERITELMGYEPEELLGRSIYEYYHALDSDHLTKTHHDMFTKGQVTTGQYRMLAK
RGGYVWVETQATVIYNTKNSQPQCIVCVNYVVSGIIQHDLIFSLQQTECVLKPVESSDMKMT
QLFTKVESEDTSSLFDKLKKEPDALTLLAPAAGDTIISLDFGSNDTETDDQQLEEVPLYNDV
MLPSPNEKLQNINLAMSPLPTAETPKPLRSSADPALNQEVALKLEPNPESLELSFTMPQIQD
QTPSPSDGSTRQSSPEPNSPSEYCFYVDSDMVNEFKLELVEKLFAEDTEAKNPFSTQDTDLD
LEMLAPYIPMDDDFQLRSFDQLSPLESSSASPESASPQSTVTVFQQTQIQEPTANATTTTAT
TDELKTVTKDRMEDIKILIASPSPTHIHKETTSATSSPYRDTQSRTASPNRAGKGVIEQTEK
SHPRSPNVLSVALSQRTTVPEEELNPKILALQNAQRKRKMEHDGSLFQAVGIGTLLQQPDDH
AATTSLSWKRVKGCKSSEQNGMEQKTIILIPSDLACRLLGQSMDESGLPQLTSYDCEVNAPI
QGSRNLLQGEELLRALDQVN

In some embodiments, the variant of HIF-1α may include HIF-1α polypeptides where a number of amino acid residues are deleted and optionally substituted by one or more amino acid residues. The variant of HIF-1α also include HIF-1α polypeptides wherein one or more amino acid residues are added at the N- or C-terminus of, or within a wild-type HIF-1α sequence. The variant of HIF-1α also may be covalently modified, for example by substitution with a moiety other than a naturally occurring amino acid or by modifying an amino acid residue to produce a non-naturally occurring amino acid.

In some embodiments, the variant of HIF-1α may be any variant forms of HIF-1α that is stable under hypoxic and non-hypoxic conditions. By “stable” it is meant that the variant of HIF-1α is more resistant to degradation under non-hypoxic conditions compared to wild-type HIF-1α (i.e., an increased half-life as compared to wild-type HIF-1α). Hypoxia is a condition where the oxygen demand in a tissue exceeds the supply of oxygen in that tissue. The terms “hypoxic” and “non-hypoxic” are understood to be relative terms with respect to oxygen concentration typically observed in a particular tissue.

In certain embodiments, the variant of HIF-1α that is stable under hypoxic and non-hypoxic conditions may comprise one or more amino acid deletions, insertions, or substitutions, particularly in the oxygen dependent degradation domain (ODD). A number of stable forms of HIF-1α with deletions in the ODD are described in U.S. Pat. No. 6,124,131, which is incorporated herein by reference.

In certain embodiments, the variant of HIF-1α that is stable under hypoxic and non-hypoxic conditions may comprise a polypeptide where one or more of hydroxylatable amino acid residue in a wild type HIF-1α is mutated such to inhibit hydroxylation of the polypeptide. Such residue may comprise P402, L562, P564 and N803, but not limited thereto.

An exemplary sequence for a stable variant of HIF-1α may be SEQ ID NO: 3.

In some embodiments, the variant of HIF-1α may comprise one or more functional HIF-1α transactivation domains. The transactivation domains are located approximately between amino acids 600 and 826 of the wild-type human HIF-1α amino acid sequence of SEQ ID NO: 1. More particularly, one transactivation domain comprises approximately amino acids 531 to 575 and a second transactivation domain comprises approximately amino acids 786 to 826. The inclusion of one or more transactivation domains in the variant of HIF-1α may be advantageous in terms of levels and spectrum of target gene expression compared to a constitutively active mutant with a heterologous transactivation domain. However, the variant of HIF-1α with a deletion, substitution or insertion in all or part of one or both of the transactivation domains or substitution of one or both of the transactivation domains with a heterologous transactivation domain may also be used.

In some embodiments, the variant of HIF-1α may be any variant forms retaining at least 50% of transactivating function of wild-type HIF-1α. The transactivating function may comprise activating transcription of erythropoietin (EPO), vascular endothelial growth factor (VEGF), glucose transporters, and glycolytic enzymes. Preferably, the variant of HIF-1α may be any variant forms retaining at least 60%, 70%, 80%, 90%, 91%, 92%, 93%, 94%, 95%, 96%, 97%, 98% or 99% of transactivating function of wild-type HIF-1α.

In some embodiments, the variant of HIF-1α may be any variant forms of HIF-1α that is stable under hypoxic and non-hypoxic conditions, and retains at least 50%, 60%, 70%, 80%, 90%, 91%, 92%, 93%, 94%, 95%, 96%, 97%, 98% or 99% of transactivating function of wild-type HIF-1α.

In some embodiments, the variant of HIF-1α may include variants any of whose residues may be changed from the corresponding residue of wild-type HIF-1α, while still retaining its transactivating functions. In some embodiments, up to 20% or more of the residues may be changed in the variant of HIF-1α compared to wild-type HIF-1α. In some embodiments, the variant of HIF-1α may be at least about 50% homologous to wild-type HIF-1α. The variant of HIF-1α that retains transactivating functions of HIF-1α includes any variant in which residues at a particular position in the sequence have been substituted by other amino acids, and further include the possibility of inserting an additional residue or residues between two residues of the wild-type HIF-1α as well as the possibility of deleting one or more residues from the wild-type HIF-1α. Any amino acid substitution, insertion, or deletion is encompassed by the present invention.

In some embodiments, the variant of HIF-1α may include minor modifications of amino acid sequence in wild-type HIF-1α or any variants described herein. The variants with minor modification may be as stable as parent protein under hypoxic and non-hypoxic conditions and/or have substantially equivalent activity as compared to the parent protein. These minor modifications may include the minor differences found in the sequence of HIF-1α isolated from different species (e.g., human, mouse, and rat HIF-1α polypeptide). Such modifications may be deliberate, as by site-directed mutagenesis, or may be spontaneous, as those found in different species. All of the variants of HIF-1α produced by these modifications are included herein as long as the biological activity (i.e., transactivating function) of HIF-1α still exists, and/or is stable under hypoxic and non-hypoxic conditions. Further, deletions of one or more amino acids can also result in modification of the structure of the resultant molecule without significantly altering its biological activity. For example, one can remove amino or carboxy terminal amino acids which are not required for HIF-1α biological activity. In some embodiment, the EV polypeptide may be selected from the group consisting of CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), PTGFRN, a fragment of the above-listed proteins, a variant of the above-listed proteins , a variant of the fragment, a fragment of the variant, and any combinations thereof, but not limited thereto. In the examples of the present invention, a fragment of PTGFRN (named as Extracellular Vesicle Sorting Motif (ESM)) was used as exosome protein.

In some embodiment, the cis-cleaving intein may be any intein known in the art. Exemplary inteins known in the art is described in Table 1. In certain embodiments, the attachment of the HIF-1α to the EV polypeptide is releasable by the cis-cleaving intein, in order to enable efficient, non-obstructed loading and endogenously triggered release of the HIF-1α into the EV. The releasable attachment between the HIF-1α and the EV polypeptide enables endogenously activatable release of the HIF-1α inside the EV and/or subsequently inside a target cell (i.e., monocyte and/or macrophage) or target tissue, to optimize loading and therapeutic activity such as anti-inflammation and/or resolution of inflammation. Truncated or in other ways optimized inteins, i.e., inteins where one or more amino acids have been removed or replaced to enhance functionality, may also be used. Without intending to be bound by any theory, it is surmised that truncation or increased-functionality mutation may increase the pH responsiveness of the intein, which further increases its utility in releasing HIF-1α from EV-based delivery system as EV may be internalized into cells via endocytosis processes. An exemplary sequence for the intein consist of SEQ ID NO: 2;

SEQ ID NO: 2
ALAEGTRIFDPVTGTTHRIEDVVGGRKPIHVVAAAKDGTLHARPVVSWF
DQGTRDVIGLRIAGGAILWATPDHKVLTEYGWRAAGELRKGDRVAQPRR
FDGFGDSAPIPARVQALADALDDKFLHDMLAEELRYSVIREVLPTRRAR
TFGLEVEELHTLVAEGVVVHAA

In some embodiment, the present invention relates to EVs comprising HIF-1α, a fragment of HIF-1α, a variant of HIF-1α, a fragment of the variant, or a variant of the fragment which is releasably attached to an intein release system, and/or HIF-1α, a fragment of HIF-1α, a variant of HIF-1a, a fragment of the variant, or a variant of the fragment that has been released from an intein release system inside the EV or inside a target cell or target organ.

In some embodiment, the polypeptide construct can be described schematically as follows (the below notation is not to be construed as illustrating any C and/or N terminal direction, it is merely meant for illustration purposes):

(HIF-1α, a fragment of HIF-1α, a variant of HIF-1α, a fragment of the variant, or a variant of the fragment)-(cis-cleaving intein)-(EV polypeptide)

In some embodiment, the polypeptide construct further comprises linker between (HIF-1α, a fragment of HIF-1α, a variant of HIF-1α, a fragment of the variant or a variant of the fragment)-(cis-cleaving intein)-(EV polypeptide).

In some embodiments, the linker may be a peptide linker. In some embodiments, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids. In some embodiments, the peptide linker may be synthetic, i.e., non-naturally occurring. In some embodiments, a peptide linker may include peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in some embodiments the peptide linker can comprise non-naturally occurring polypeptides which are modified forms of naturally occurring polypeptides (e.g., comprising a mutation such as an addition, substitution, or deletion). Linkers can be susceptible to cleavage (“cleavable linker”) thereby facilitating release of the exogenous biologically active molecule. In some embodiments, the linker may comprise a non-cleavable linker.

In some embodiments, the polypeptide construct may be located or positioned in/on the membrane of EVs. In some embodiment, of the polypeptide construct, the EV polypeptide may be located or positioned in the membrane of EVs at least in part. In some embodiment, of the polypeptide construct, the HIF-1α, a fragment of HIF-1α, a variant of HIF-1α, a fragment of the variant or a variant of the fragment may be located or positioned on the membrane and/or inside the membrane of EVs. In some embodiments, the HIF-1α, a fragment of HIF-1α, a variant of HIF-1α, a fragment of the variant, or a variant of the fragment may be expressed or displayed or presented on the membrane of EVs.

In some embodiments, the EVs may further comprise at least one therapeutically active payload on the surface of the EVs, inside the EVs, or both. In some embodiments, the therapeutically active payload may be selected from the group consisting of nucleotides, amino acids, peptides, proteins lipids, carbohydrates, and small molecules, but not limited thereto. In some embodiments, non-limiting examples of other suitable therapeutically active payload includes pharmacologically active drugs and genetically active molecules, including anti-cancer agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, which are incorporated herein by reference. Suitable payloads further include toxins, and biological and chemical warfare agents, for example see Somani, S. M. (ed.), Chemical Warfare Agents, Academic Press, New York (1992)), which is incorporated herein by reference.

In some certain embodiments, the EVs may further comprise at least one anti-inflammatory agent. The exemplary anti-inflammatory agent may include, but are not limited to curcumin, non-steroidal anti-inflammatory drugs (NSAIDs) including, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, dec ano ate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium. In addition, cytokine antagonists, such as aptamers of IL-10, IL-6, IL-8, TNF-alpha (TNF-α), IL-5, IL-13, TGF-beta (TGF-β), VEGF and etc., may be used for anti-inflammatory effects.

In some embodiments, the EVs may further comprise at least one targeting moiety. In some embodiments, the targeting moiety can be used for targeting the EVs to a specific organ, tissue, or cell for a treatment using the EVs. In a certain embodiment, the targeting moiety may bind to a marker (or target molecules) expressed on a cell or a population of cells. In certain embodiments, the marker may be expressed on multiple cell types, e.g., all antigen-present cells (e.g., dendritic cells, macrophages, and B lymphocytes). In some embodiments, the marker may be expressed only on a specific population of cells (e.g., dendritic cells). Non-limiting examples of markers that are expressed on specific population of cells (e.g., dendritic cells) include a C-type lectin domain family 9 member A (CLEC9A) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), CD45, CD64, CD11b, F4/80, Ly6C and any combination thereof. In some embodiments, the targeting moiety may be an antibody or antigen-binding fragment thereof. Antibodies and antigen-binding fragments thereof include whole antibodies, polyclonal, monoclonal and recombinant antibodies, fragments thereof, and they may further include single-chain antibodies, humanized antibodies, murine antibodies, chimeric, mouse-human, mouse-primate, primate-human monoclonal antibodies, anti-idiotype antibodies, antibody fragments (e.g., scFv, (scFv)2, Fab, Fab′, and F(ab′)2, F(ab1)2, Fv, dAb, and Fd fragments), diabodies, and antibody-related polypeptides. Antibodies and antigen-binding fragments thereof may include bispecific antibodies and multispecific antibodies so long as they exhibit the desired biological activity or function. In a preferred embodiment, the targeting moiety may target monocyte and/or monocyte-derived macrophage. In a preferred embodiment, the targeting moiety may target CD45+CD64−CD11b+F4/80^{low} monocyte and/or CD45+CD64+CD11b+F4/80+ monocyte-derived macrophage. In some embodiment, the targeting moiety may be comprised in the polypeptide construct. In some embodiment, the targeting moiety may be linked to the EV polypeptide that is comprised in the polypeptide construct directly or via a linker.

In some embodiment, the polypeptide construct comprising at least one targeting moiety can be described schematically as follows (the below notation is not to be construed as illustrating any C and/or N terminal direction, it is merely meant for illustration purposes):

(HIF-1α, a fragment of HIF-1α, a variant of HIF-1α, a fragment of the variant or a variant of the fragment)-(cis-cleaving intein)-(EV polypeptide)-(targeting moiety)

In some embodiments, the polypeptide construct may further comprise at least one fusogenic peptide. In some embodiment, the fusogenic peptide may induce a homologous or target fusion between cells or membrane vesicles surrounded by a plasma membrane. The fusogenic peptide as such may include (GALA)n (n is an integer from 1 to 10), (KALA)n (n is an integer from 1 to 10), INF7, influenza virus HA2, melittin, octa-arginine (R8) peptide, vesicular stomatitis virus glycoprotein (VSV-G), tat protein of HIV, HSV-1 gB, EBV gB, thgoto virus G protein, AcMNPV gp64, a fragment thereof, a variant thereof, a variant of the fragment and a fragment of the variant and any combinations thereof, but numerous other peptides capable of transporting a polypeptide construct to EVs are comprised within the scope of the present invention.

In some embodiments, the polypeptide construct comprising at least one fusogenic peptide may deliver HIF-1α more efficiently to Evs as compared to a polypeptide construct without the fusogenic peptide. In some embodiments, the amount of delivered HIF-1α to EVs using the polypeptide construct comprising the fusogenic peptide may be increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% or more, compared to the amount of delivered HIF-1α to EVs using a polypeptide construct without the fusogenic peptide.

In some embodiments, the polypeptide construct comprising at least one fusogenic peptide can be described schematically as follows (the below notation is not to be construed as illustrating any C and/or N terminal direction, it is merely meant for illustration purposes):

(HIF-1α, a fragment of HIF-1α, a variant of HIF-1α, a fragment of the variant or a variant of the fragment)-(cis-cleaving intein)-(EV polypeptide)-(fusogenic peptide)

In some embodiments, the fusogenic peptide may be linked to the EV polypeptide that is comprised in the polypeptide construct directly or via a linker.

In some embodiments, the polypeptide construct comprising at least one fusogenic peptide and at least one targeting moiety can be described schematically as follows (the below notation is not to be construed as illustrating any C and/or N terminal direction, it is merely meant for illustration purposes):

(HIF-1α, a fragment of HIF-1α, a variant of HIF-1α, a fragment of the variant or a variant of the fragment)-(cis-cleaving intein)-(EV polypeptide)-(fusogenic peptide)-(targeting moiety)

In some embodiments, the EVs comprising the polypeptide construct may be prepared by the method comprising the steps of (1) introducing into a producer cell at least one polynucleotide construct (e.g., a vector) which encodes the polypeptide construct, and (2) expressing the corresponding polypeptide(s) from the polynucleotide construct(s). Typically, the method may further comprise a step (3) of collecting the EVs generated (i.e., released) by the producer cell, optionally followed by purification and/or isolation. The introduction of suitable polynucleotide constructs into a producer cell (typically a cell culture comprising a suitable EV-producing cell type for production of EVs) may be achieved using a variety of conventional techniques, such as transfection, virus-mediated transformation, electroporation, etc. Transfection may be carried out using conventional transfection reagents such as liposomes, CPPs, cationic lipids or polymers, calcium phosphates, dendrimers, etc. Virus-mediated transfection is also a highly suitable methodology, and it may be carried out using conventional virus vectors such as adenoviral or lentiviral vectors.

In some embodiments, the subject may be mammals and non-mammals. Examples of mammals include, but are not limited to, humans, chimpanzees, apes monkeys, cattle, horses, sheep, goats, swine, rabbits, dogs, cats, rats, mice, guinea pigs, and the like. Examples of non-mammals include, but are not limited to, birds, fishes, and the like.

In some embodiments, the subject may be in status of increased MI and/or M1-like macrophages compared to normal condition. The administering of EV described herein to the subject may alleviate any abnormal condition or symptom caused by increased MI and/or M1-like macrophages.

In some embodiments, the subject may be in inflammatory condition. The administering of EV described herein to the subject may alleviate the inflammatory condition.

In some embodiments, the subject may be in inflammatory condition accompanying increased proportion and/or increased number of monocyte-derived macrophage compared to normal condition. The administering of EV described herein to the subject may alleviate any abnormal condition or symptom caused by increased proportion and/or increased number of monocyte-derived macrophages.

In some embodiments, the administering of EV described herein may modify polarization of macrophages to favor resolutive macrophages and/or modify balance between the different subtypes of macrophages toward resolutive macrophages and/or inducing differentiation of monocytes to resolutive macrophages and/or inducing phenotype switching from macrophages to resolutive macrophages in the subject as compared to the same subject prior to the treatment of the EV described herein.

In some embodiments, the resolutive macrophage is selected from the group consisting of an Ly6C(low) macrophage and a MerTK-positive macrophage.

In some embodiments, the resolutive macrophage is selected from the group consisting of a pro-efferocytic macrophage and an anti-oxidant macrophage.

In some embodiments, the pro-efferocytic macrophage is a macrophage expressing at least one marker selected from the group consisting of Ly6c2^{low}, MerTK, Marco, Stab 1, Arg1, Nr1h3, id1/3, Hmox1, il1rn, Ccl6, Ccl12, Mmp14. In some embodiments, the pro-efferocytic macrophage is a macrophage expressing at least two, three, four, five, six, seven, eight, nine, ten, eleven or twelve markers selected from the group consisting of Ly6c2^{low}, MerTK, Marco, Stab1, Arg1, Nr1h3, id1/3, Hmox1, il1rn, Ccl6, Ccl12, Mmp14. In a preferred embodiment, the pro-efferocytic macrophage is an Ly6c2^{low}, MerTK+, Marco+, Stab1+, Arg1+, Nr1h3+, id1/3+, Hmox1+, il1rn+, Ccl6+, Ccl12+and Mmp14+ macrophage.

In some embodiments, the anti-oxidant macrophage is a macrophage expressing at least one marker selected from the group consisting of Ly6c2^{low}, C/EBPβ, Nr4a1, Pparg, Stat3, Nfe212, Bcl2, Csf1r, il1b, il17ra, Pecam1, ifngr1. In some embodiments, the anti-oxidant macrophage is a macrophage expressing at least two, three, four, five, six, seven, eight, nine, ten, eleven or twelve markers selected from the group consisting of Ly6c2^{low}, C/EBPβ, Nr4a1, Pparg, Stat3, Nfe212, Bcl2, Csf1r, il1b, il17ra, Pecam1, ifngr1. In a preferred embodiment, the anti-oxidant macrophage is an Ly6c2^{low}, C/EBPβ+, Nr4a1+, Pparg+, Stat3+, Nfe212+, Bcl2+, Csf1r+, il1b+, il17ra+, Pecam1+ and ifngr1+ macrophage.

In a further aspect, the present invention provides a resolutive macrophage-inducing agent, comprising extracellular vesicles (EVs) comprising at least one polypeptide construct comprising: (i) HIF-1α (Hypoxia-inducible factor 1-alpha), a fragment of HIF-1α, a variant of HIF-1α, a fragment of the variant or a variant of the fragment; (ii) at least one EV polypeptide; and (iii) a monomeric cis-cleaving intein.

As for the EV and polypeptide construct, the above description may be applied in the same manner.

In a still further aspect, the present invention provides a method for preventing or treating inflammatory diseases, conditions, or symptoms, the method comprising administering to a subject in need thereof a prophylactically or therapeutically effective amount of extracellular vesicle (EV) comprising at least one polypeptide construct comprising: (i) HIF-1α, a fragment of HIF-1α, a variant of HIF-1α, a fragment of the variant, or a variant of the fragment; (ii) at least one EV polypeptide; and (iii) a monomeric cis-cleaving intein.

As for the EV and polypeptide construct, the above description may be applied in the same manner.

In some embodiments, the inflammatory disease, condition, or symptom is related to acute and/or chronic disease, condition, or symptom selected from the group consisting of single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, non-alcoholic steatohepatitis(NASH), inflammatory arthritis, gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, Crohn's and ulcerative colitis, terminal ileitis, alcoholic steatohepatitis, hepatotoxicity, liver infection, remote liver injury, lupus, autoimmune diseases associated with acute or chronic inflammation (e.g., rheumatoid arthritis), and acute or chronic inflammation associated with viral, bacterial or fungal infection, but not limited thereto (Gwag T, et al., Anti-CD47 antibody treatment attenuates liver inflammation and fibrosis in experimental non-alcoholic steatohepatitis models. Liver Int, 2022. 42(4): p. 829-841; Cui L, et al., Activation of JUN in fibroblasts promotes pro-fibrotic programme and modulates protective immunity. Nat Commun, 2020. 11(1): p. 2795; Lerbs T, et al., CD47 prevents the elimination of diseased fibroblasts in scleroderma. JCI Insight, 2020. 5(16); Gerlinde W, et al., Unifying mechanism for different fibrotic diseases. Proc Natl Acad Sci USA, 2017. 114(18):4757-4762; Kojima Y, et al. CD47-blocking antibodies restore phagocytosis and prevent atherosclerosis. Nature. 2016. 536(7614):86-90; Kai-Uwe J, et al., Effect of CD47 Blockade on Vascular Inflammation. N Engl J Med. 2021. 384(4):382-383; Cham L B, et al. Immunotherapeutic Blockade of CD47 Inhibitory Signaling Enhances Innate and Adaptive Immune Responses to Viral Infection. Cell Rep. 2020. 31(2):107494; McLaughlin K M, et al., A Potential Role of the CD47/SIRPalpha Axis in COVID-19 Pathogenesis. Curr Issues Mol Biol. 2021 22;43(3):1212-1225; H Zhao, et al., Role of necroptosis in the pathogenesis of solid organ injury. Cell Death Dis. 2015. 19;6(11); M Deutsch, et al., Divergent effects of RIP1 or RIP3 blockade in murine models of acute liver injury. Cell Death Dis. 2015. 6(5):e1759, which are incorporated herein by reference.).

In some certain embodiments, the organ failure is selected from the group consisting of acute liver failure, bone marrow failure, acute kidney failure, and acute heart failure, but not limited thereto.

In some certain embodiments, the viral infection is selected from the group consisting of hepatitis virus infection, ZIKA virus infection, herpes virus infection, papillomavirus infection, influenza virus infection, coronavirus infection, COVID-19, and SARS, but not limited thereto.

In some certain embodiments, the fibrosis is selected from the group consisting of pulmonary fibrosis, cystic fibrosis, idiopathic pulmonary fibrosis, skin fibrosis, kidney fibrosis, bone marrow fibrosis, interstitial pulmonary fibrosis, liver fibrosis, bridging fibrosis of the liver, arthrofibrosis, keloid fibrosis, mediastinal fibrosis, myelofibrosis, myocardial fibrosis, nephrogenic systemic fibrosis, progressive massive fibrosis, retroperitoneal fibrosis, and stromal fibrosis, but not limited thereto.

In some embodiments, the EV may be administered orally or parenterally. When administered parenterally, the pharmaceutical composition may be administered via various routes, including intravenous administration, intra-arterial administration, epidural administration, intracerebral administration, intracerebroventricular administration, nasal administration, intramuscular administration, intraperitoneal administration, subcutaneous administration, intradermal administration, transdermal absorption, etc.

In some embodiments, the inflammatory diseases, conditions, or symptoms are related to the increased proportion and/or increased number of monocyte-derived macrophage compared to normal condition. In some embodiment, the inflammatory diseases, conditions, or symptoms are related to the increased proportion and/or increased number of monocyte and monocyte-derived macrophage compared to normal condition. The administration of the EVs described herein may cause increase of at least one type of macrophages selected from the group consisting of pro-efferocytic macrophages, anti-oxidant macrophages, Ly6C(low) macrophages, and MerTK-positive macrophages, resulting in resolution of inflammation.

In a still further aspect, the present invention provides a pharmaceutical composition for preventing or treating inflammatory diseases, conditions, or symptoms, the composition comprising extracellular vesicles (EVs) comprising at least one polypeptide construct comprising: (i) HIF-1α, a fragment of HIF-1α, a variant of HIF-1α, a fragment of the variant, or a variant of the fragment; (ii) at least one EV polypeptide; and; (iii) a monomeric cis-cleaving intein.

The composition may further comprise a pharmaceutically acceptable carrier and/or excipient. Pharmaceutically acceptable excipients or carriers can be determined in part by the particular composition being administered, as well as by the particular method used to administer the composition. Accordingly, there is a wide variety of suitable formulations of pharmaceutical compositions. See, e.g., Remington's Pharmaceutical Sciences, Mack Publishing Co., Easton, Pa. 21st ed. (2005), which is incorporated herein by reference. The pharmaceutical compositions can be generally formulated sterile and in full compliance with all Good Manufacturing Practice (GMP) regulations of the U.S. Food and Drug Administration.

In some embodiments, a pharmaceutically acceptable carrier may be various oral or parenteral formulations. For the preparation of formulations, a diluent or excipient such as a filler, an extender, a binder, a humectant, a disintegrant, a surfactant, etc., may be used. Solid formulations for oral administration may include tablets, pills, powders, granules, capsules, etc., and these solid formulations may be prepared by adding at least one excipient, e.g., starch, calcium carbonate, sucrose or lactose, gelatin, etc. Additionally, lubricants, such as magnesium stearate, talc, etc., may be used, in addition to the simple excipient. Liquid formulations for oral administration may include suspensions, liquid medicines for internal use, emulsions, syrups, etc., and various excipients such as humectants, sweeteners, fragrances, and preservatives, may be used, in addition to the frequently used simple diluents such as water and liquid paraffin. Formulations for parenteral administration may include sterile aqueous solutions, non-aqueous solvents, suspensions, emulsions, lyophilized formulations, suppositories, etc. Examples of the non-aqueous solvents and suspensions may include vegetable oils such as propylene glycol, polyethylene glycol, and olive oil; an injectable ester such as ethyl oleate; etc. Examples of the bases for suppositories may include Witepsol, macrogol, Tween 61, cacao butter, laurinum, glycerogelatin, etc.

In some embodiments, the pharmaceutical composition may have one formulation selected from the group consisting of tablets, pills, powders, granules, capsules, suspensions, solutions, emulsions, syrups, sterilized aqueous solutions, non-aqueous solutions, lyophilized formulations, and suppositories.

In some embodiments, the pharmaceutical composition may be administered orally or parenterally. When administered parenterally, the pharmaceutical composition may be administered via various routes, including intravenous administration, intra-arterial administration, epidural administration, intracerebral administration, intracerebroventricular administration, nasal administration, intramuscular administration, intraperitoneal administration, subcutaneous administration, intradermal administration, transdermal absorption, etc.

In some embodiments, the pharmaceutical composition may be administered in a therapeutically effective amount.

In some embodiments, the pharmaceutical composition may be administered as an individual therapeutic agent, in combination with other therapeutic agents for inflammatory diseases, or sequentially or simultaneously with a conventional therapeutic agent(s) and may be administered once or multiple times. It is important to administer an amount to obtain the maximum effect with a minimum amount without adverse effects considering all of the factors, and these factors can easily be determined by one of ordinary skill in the art.

In a still further aspect, the present invention provides an extracellular vesicle (EV) comprising at least one polypeptide construct comprising: (i) at least one fusogenic peptide; (ii) at least one polypeptide of interest (PoI); (iii) at least one EV polypeptide; and (iv) a monomeric cis-cleaving intein,.

As for the EV, polypeptide construct, fusogenic peptide, EV polypeptide and monomeric cis-cleaving intein, the above description may be applied in the same manner.

In some embodiments, the at least one PoI is releasably attached to an EV polypeptide. The attachment of the PoI to the EV polypeptide is releasable, in order to enable efficient, non-obstructed loading and endogenously triggered release of the therapeutic polypeptide of interest into the EV.

In some embodiments, the polypeptide construct comprising at least one fusogenic peptide may deliver PoI more efficiently to EVs as compared to a polypeptide construct without fusogenic peptide. In some embodiments, the amount of delivered PoI to EVs using the polypeptide construct comprising the fusogenic peptide may be increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% or more, compared to the amount of delivered PoI to EVs using a polypeptide construct without the fusogenic peptide.

In some embodiment, the polypeptide construct can be described schematically as follows (the below notation is not to be construed as illustrating any C and/or N terminal direction, it is merely meant for illustration purposes):

(PoI)-(cis-cleaving intein)-(EV polypeptide)-(fusogenic peptide)

The present invention relates to EV-based therapeutics comprising essentially any polypeptide of interest (PoI), typically for therapeutic or prophylactic purposes but potentially also for cosmetic uses. The PoI—or PoIs in the cases where a plurality (i.e., more than one) PoI are utilized—may be any suitable polypeptide, that is any molecule comprising a plurality of amino acids, i.e., a protein or a peptide. The PoI may be selected from anyone of the following non-limiting examples of therapeutic, prophylactic, or cosmetic polypeptides: antibodies, intrabodies, single chain variable fragments (scFv), affibodies, bi-och multi specific antibodies or binders, receptors, ligands, enzymes such as enzymes lacking and/or defect in lysosomal storage diseases (LSDs), tumor suppressors such as p53, pVHL, APC, CD95, ST5, YPEL3, ST7, and ST14, viral or bacterial inhibitors, cell component proteins, DNA and/or RNA binding proteins, nucleases such as Cas, Cas9, and Cpf1, proteinases, integrases, transcription factors, growth factors, apoptosis inhibitors and inducers, structural proteins, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. The fact that EVs enable reaching the intracellular milieu in a highly efficient manner means that a vast number of intracellular targets becomes druggable. Thus, a therapeutic protein of interest (PoI) is typically either a protein that binds to an intracellular target (for instance an intrabody against an oncogenic protein such as c-Myc or a decoy receptor binding its intracellular interaction partner) or a PoI that is meant to exert a desired effect intracellularly (the PoI may for instance be dystrophin as a treatment of Duchenne's muscular dystrophy (DMD), a PoI for replacement of a missing or defect protein (such an enzyme like NPC1, GBA, or AGAL, etc. for enzyme replacement therapy, the Huntingtin protein or BDNF for the treatment of e.g. Huntington's disease or other neurodegenerative disorders), a tumor suppressor such as p53 for treatment of cancer, or an NFkB inhibitor for treatment of inflammatory diseases. Targets of interest for intrabodies delivered with the aid of the EVs of the present invention may include pathological forms of alpha-synuclein, LRRK2, Tau, Beta amyloid, APP, C9orf72, SOD1, TDP43, FUS and prion proteins. One class of Pols with considerable therapeutic potential are the RNA-binding proteins (RBPs), which may be used to aid intracellular delivery of RNA therapeutics such as mRNA, RNAi agents such as short-hairpin RNA or microRNA, or antisense agents for splice-switching or silencing. Non-limiting examples of RNA-binding proteins are hnRNPA1, hnRNPA2B1, DDX4, ADAD1, DAZL, ELAVL4, IGF2BP3, SAMD4A, TDP43, FUS, FMR1, FXR1, FXR2, EIF4A1-3, the MS2 coat protein, as well as any domains, parts or derivates, thereof. More broadly, particular subclasses of RNA-binding proteins and domains, e.g., mRNA binding proteins (mRBPs), pre-rRNA-binding proteins, tRNA-binding proteins, small nuclear or nucleolar RNA-binding proteins, non-coding RNA-binding proteins, and transcription factors (TFs). Furthermore, various domains and derivatives may also be used as the PoI for transport of an RNA cargo. Non-limiting examples of RNA-binding PoI include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and Ribosomal_L7Ae. Such RNA-binding domains may be present in a plurality, alone or in combination with others, and may also form part of a larger RNA-binding protein construct as such, as long as their key function (i.e., the ability to transport an RNA cargo of interest, e.g., an mRNA or a short RNA) is maintained.

In some embodiments, the EVs of the present invention further comprise at least one targeting moiety. As for the targeting moiety, the above description may be applied in the same manner.

In a still further aspect, the present invention provides a highly effective method for producing EVs with strong therapeutic efficacy in large quantities. The methods of the present invention comprise the steps of (a) introducing into a producer cell at least one polynucleotide construct which encodes a polypeptide construct comprising at least one fusogenic peptide, at least one polypeptide of interest (PoI), at least one EV polypeptide and a monomeric cis-cleaving intein and (b) expressing the corresponding polypeptide construct from the polynucleotide construct. Typically, the method may further comprise a step (c) of collecting the EVs generated (i.e., released) by the producer cell, into which EVs the PoI has been released through endogenous triggering of the monomeric cis-cleaving intein.

In a still further aspect, the present invention provides polynucleotide and polypeptide constructs. The polynucleotide constructs of the present invention typically comprise nucleotide stretches encoding a polypeptide construct comprising at least one fusogenic peptide, at least one polypeptide of interest (PoI), at least one EV polypeptide and a monomeric cis-cleaving intein. Thus, the present invention naturally also relates to the corresponding polypeptide constructs, i.e., a polypeptide construct comprising at least one fusogenic peptide, at least one polypeptide of interest (PoI), at least one EV polypeptide and a monomeric cis-cleaving intein. Furthermore, the present invention also provides EV-producing cells (typically cells present in the form of cell culture but also individual cells as such) comprising the above-mentioned polynucleotide construct(s) and/or the above-mentioned polypeptide(s).

In a still further aspect, the present invention provides methods for delivery of a PoI into the intracellular environment or into the membrane of a target cell, either in vitro or in vivo. The methods comprise contacting a target cell with an EV comprising either (i) a PoI which is releasably attached to an EV polypeptide using a monomeric cis-cleaving intein or (ii) a PoI which has been released from an EV polypeptide, either inside the EV or inside the target cell. Importantly, the PoIs of the present invention are delivered into target cells in a substantially unconjugated form that could potentially hamper the activity of the Pot And the PoIs of the present invention are delivered in a significantly increased amount by at least one fusogenic peptide compared to the previously disclosed methods in the art.

In certain embodiments, the PoI may be an integral membrane protein conjugated to an EV polypeptide with the aid of the monomeric cis-cleaving intein. An integral membrane PoI may be presented on the outer, inner or both surfaces of an EV. Without wishing to be bound by any theory, it is surmised that following uptake into a target cell, an EV—comprising a polypeptide construct which in turn comprises an integral membrane PoI—is trafficked to the endoplasmic reticulum (ER). The contents of the EV, i.e., the polypeptide construct comprising the integral membrane PoI, may be processed and sorted at the ER followed by ER-mediated trafficking to the appropriate compartment of the target cell. Thus, a polypeptide construct comprising an EV protein conjugated with the aid of a monomeric cis-cleaving intein to an integral plasma membrane protein (for instance a G-protein coupled receptor (GPCR)) would be routed to the plasma membrane of a target cell, whereas a membrane protein natively present in a lysosomal membrane (i.e., a lysosomal membrane protein) would be routed to a lysosome of the target cell.

The release of the PoI is as above-outlined mediated by a monomeric cis-cleaving intein which is fused to the PoI and/or to the EV polypeptide. In an advantageous embodiment, the polynucleotide construct encoding for the subsequent polypeptide construct is designed in such a way so as to place the monomeric cis-cleaving intein in between the PoI and the EV polypeptide. This arrangement enables easy manufacturing of the constructs and efficient release of the PoI in the desired location.

In a still further aspect, the present invention provides pharmaceutical compositions comprising EVs in accordance with the present invention. Typically, the pharmaceutical compositions of the present invention comprise at least one type of therapeutic EV (i.e., a population of EVs having comprising a certain desired PoI) formulated with at least one pharmaceutically acceptable excipient.

In a still further aspect, the present invention provides cosmetic applications of the EVs comprising PoIs. Thus, the present invention pertains to skin care products such as creams, lotions, gels, emulsions, ointments, pastes, powders, liniments, sunscreens, shampoos, etc., comprising a suitable EV, in order to improve and/or alleviate symptoms and problems such as dry skin, wrinkles, folds, ridges, and/or skin creases.

In a still further aspect, the present invention provides EVs of the present invention for use in medicine. Naturally, when an EV comprising a PoI in accordance with the present invention is used in medicine, it is in fact normally a population of EVs that is being used.

The EVs of the present invention may be used for prophylactic and/or therapeutic purposes, e.g., for use in the prophylaxis and/or treatment and/or alleviation of various diseases and disorders. A non-limiting sample of diseases wherein the EVs as per the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, kidney failure, graft-vs-host disease, Duchenne's muscular dystrophy and other muscular dystrophies, lysosomal storage diseases such as Gaucher disease, Fabry's disease, MPS I, II (Hunter syndrome), and III, Niemann-Pick disease, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers.

### EXAMPLES

Hereinafter, embodiments of the present disclosure will be described in detail with the following examples. However, the present disclosure is not limited to the examples explained. Rather, the examples are provided to sufficiently transfer the concept of the present disclosure to a person skilled in the art to thorough and complete contents introduced herein.

### Example 1

### Construction of Plasmid DNAs

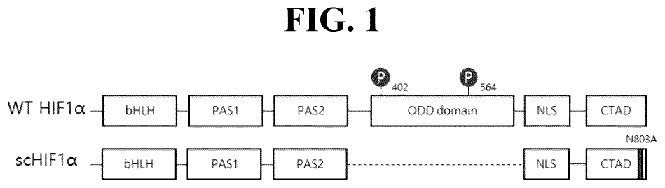

The difficulty in transmitting or stabilizing HIF1α is due to its nature as a transcription factor. Transcription factors regulate gene expression within cells, hence they are stabilized or produced only in specific environments and then move into the nucleus. Under normal oxygen concentrations, HIF1α's ODD domain undergoes phosphorylation, leading to its degradation. To overcome this, the ODD domain of HIFla was removed to ensure stable functioning even in normal oxygen concentrations. In addition, to maximize its efficacy as a transcription factor, the 803rd amino acid of HIF1α, asparagine, was mutated to alanine to create scHIF1α (FIG. 1). This modification is based on the mechanism where the factor inhibiting HIF suppresses the action of HIF1α. Therefore, scHIF1α possesses a structure that maintains stability and activation even under normal oxygen concentrations.

scHIF1α Sequence (SEQ ID NO: 3):
MEGAGGANDKKKISSERRKEKSRDAARSRRSKESEVFYELAHQLPLPHNVSSHLDKASVMRL
TISYLRVRKLLDAGDLDIEDDMKAQMNCFYLKALDGFVMVLTDDGDMIYISDNVNKYMGLTQ
FELTGHSVFDFTHPCDHEEMREMLTHRNGLVKKGKEQNTQRSFFLRMKCTLTSRGRIMNIKS
ATWKVLHCTGHIHVYDTNSNQPQCGYKKPPMTCLVLICEPIPHPSNIEIPLDSKTELSRHSL
DMKFSYCDERITELMGYEPEELLGRSIYEYYHALDSDHLTKTHHDMFTKGQVTTGQYRMLAK
RGGYVWVETQATVIYNTKNSQPQCIVCVNYVVSGIIQHDLIFSLQQTECVLKPVESSDMKMT
QLFTKVESEDTSSLFDKLKKEPDDTDLDLEMLAPYIPMDDDFQLRSFDQLSPLESSSASPES
ASPQSTVTVFQQTQIQEPTANATTTTATTDELKTVTKDRMEDIKILIASPSPTHIHKETTSA
TSSPYRDTQSRTASPNRAGKGVIEQTEKSHPRSPNVLSVALSQRTTVPEEELNPKILALQNA
QRKRKMEHDGSLFQAVGIGTLLQQPDDHAATTSLSWKRVKGCKSSEQNGMEQKTIILIPSDL
ACRLLGQSMDESGLPQLTSYDCEVAAPIQGSRNLLQGEELLRALDQVN

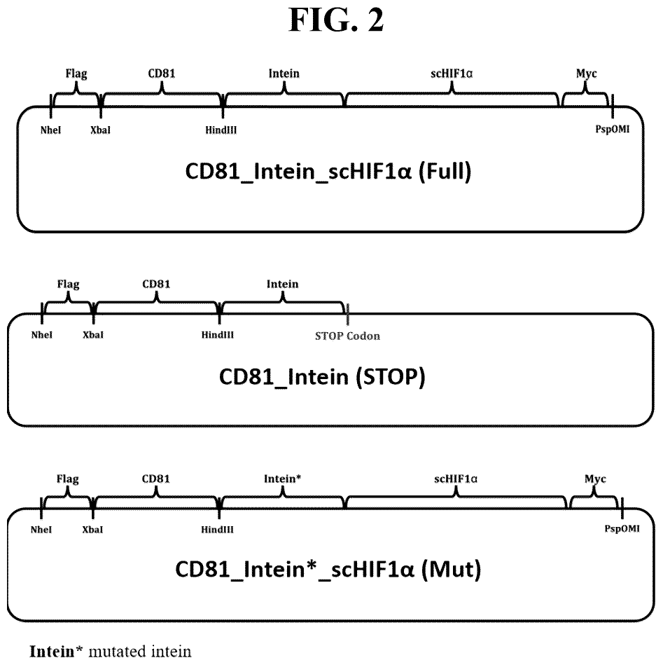

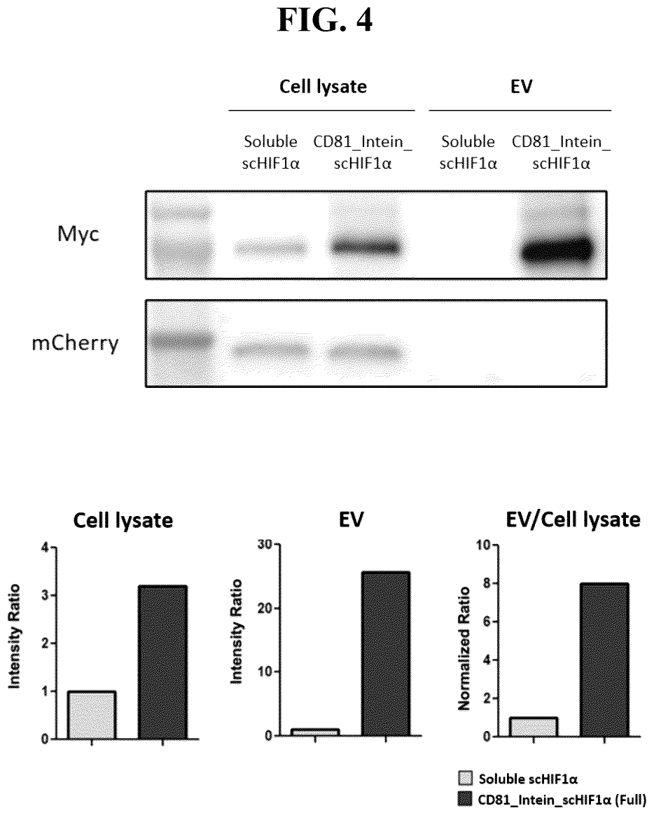

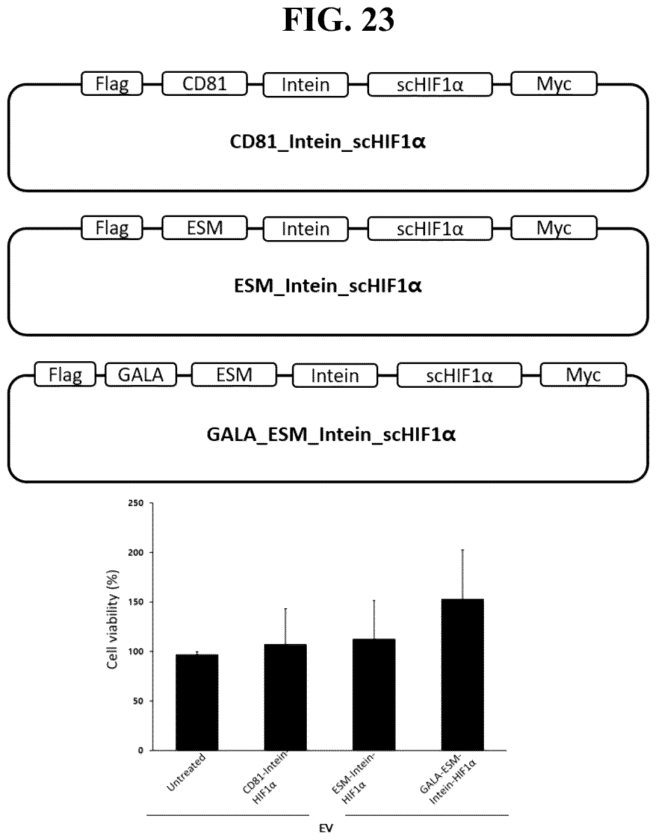

According to some embodiments of the invention, a DNA construct capable of effectively encapsulating the scHIF1α protein within EVs was created. The conventional plasma DNA was purchased from Invitrogen, and additional vector construction for the desired plasma DNA sequence was achieved using a cloning method with a restriction enzyme and an in-fusion cloning method of Takara's products. The vector was created based on pcDNA3.1. To clarify the loading function of intein in the EV, a total of three plasmids, CD81_Intein_scHIF1α (Full), CD81_Intein (Stop), and CD81_Intein*_scHIF1α (Mut), were created (FIG. 2). To mitigate variables associated with transfection efficiency, we utilized the mCherry plasmid (FIG. 4). To verify the increased delivery efficiency of therapeutic molecules loaded in the EV to the target location within cells due to GALA, GALA_CD81_Intein_scHIF1α was created (FIG. 22).

CD81_Intein_scHIF1α (Full) Sequence (SEQ ID NO: 4):
MDYKDDDDKSRGVEGCTKCIKYLLFVFNFVFWLAGGVILGVALWLRHDPQTTNLLYLELGDK
PAPNTFYVGIYILIAVGAVMMFVGFLGCYGAIQESQCLLGTFFTCLVILFACEVAAGIWGFV
NKDQIAKDVKQFYDQALQQAVVDDDANNAKAVVKTFHETLDCCGSSTLTALTTSVLKNNLCP
SGSNIISNLFKEDCHQKIDDLFSGKLYLIGIAAIVVAVIMIFEMILSMVLCCGIRNSSVYKL
ALAEGTRIFDPVTGTTHRIEDVVGGRKPIHVVAAAKDGTLHARPVVSWFDQGTRDVIGLRIA
GGAILWATPDHKVLTEYGWRAAGELRKGDRVAQPRREDGFGDSAPIPARVQALADALDDKFL
HDMLAEELRYSVIREVLPTRRARTFGLEVEELHTLVAEGVVVHNCMEGAGGANDKKKISSER
RKEKSRDAARSRRSKESEVFYELAHQLPLPHNVSSHLDKASVMRLTISYLRVRKLLDAGDLD
IEDDMKAQMNCFYLKALDGFVMVLTDDGDMIYISDNVNKYMGLTQFELTGHSVFDFTHPCDH
EEMREMLTHRNGLVKKGKEQNTQRSFFLRMKCTLTSRGRIMNIKSATWKVLHCTGHIHVYDT
NSNQPQCGYKKPPMTCLVLICEPIPHPSNIEIPLDSKTFLSRHSLDMKFSYCDERITELMGY
EPEELLGRSIYEYYHALDSDHLTKTHHDMFTKGQVTTGQYRMLAKRGGYVWVETQATVIYNT
KNSQPQCIVCVNYVVSGIIQHDLIFSLQQTECVLKPVESSDMKMTQLFTKVESEDTSSLEDK
LKKEPDDTDLDLEMLAPYIPMDDDFQLRSFDQLSPLESSSASPESASPQSTVTVFQQTQIQE
PTANATTTTATTDELKTVTKDRMEDIKILIASPSPTHIHKETTSATSSPYRDTQSRTASPNR
AGKGVIEQTEKSHPRSPNVLSVALSQRTTVPEEELNPKILALQNAQRKRKMEHDGSLFQAVG
IGTLLQQPDDHAATTSLSWKRVKGCKSSEQNGMEQKTIILIPSDLACRLLGQSMDESGLPQL
TSYDCEVAAPIQGSRNLLQGEELLRALDQVNEFEQKLISEEDL
CD81_Intein (Stop) Sequence (SEQ ID NO: 5):
MDYKDDDDKSRGVEGCTKCIKYLLFVFNFVFWLAGGVILGVALWLRHDPQTTNLLYLELGDK
PAPNTFYVGIYILIAVGAVMMFVGFLGCYGAIQESQCLLGTFFTCLVILFACEVAAGIWGFV
NKDQIAKDVKQFYDQALQQAVVDDDANNAKAVVKTFHETLDCCGSSTLTALTTSVLKNNLCP
SGSNIISNLFKEDCHQKIDDLESGKLYLIGIAAIVVAVIMIFEMILSMVLCCGIRNSSVYKL
ALAEGTRIFDPVTGTTHRIEDVVGGRKPIHVVAAAKDGTLHARPVVSWFDQGTRDVIGLRIA
GGAILWATPDHKVLTEYGWRAAGELRKGDRVAQPRRFDGFGDSAPIPARVQALADALDDKFL
HDMLAEELRYSVIREVLPTRRARTFGLEVEELHTLVAEGVVVHNC
CD81_Intein*_scHIF1α (Mut) Sequence (SEQ ID NO: 6):
MDYKDDDDKSRGVEGCTKCIKYLLFVFNFVFWLAGGVILGVALWLRHDPQTTNLLYLELGDK
PAPNTFYVGIYILIAVGAVMMFVGFLGCYGAIQESQCLLGTFFTCLVILFACEVAAGIWGFV
NKDQIAKDVKQFYDQALQQAVVDDDANNAKAVVKTFHETLDCCGSSTLTALTTSVLKNNLCP
SGSNIISNLFKEDCHQKIDDLESGKLYLIGIAAIVVAVIMIFEMILSMVLCCGIRNSSVYKL
ALAEGTRIFDPVTGTTHRIEDVVGGRKPIHVVAAAKDGTLHARPVVSWFDQGTRDVIGLRIA
GGAILWATPDHKVLTEYGWRAAGELRKGDRVAQPRREDGFGDSAPIPARVQALADALDDKFL
HDMLAEELRYSVIREVLPTRRARTFGLEVEELHTLVAEGVVVHAAMEGAGGANDKKKISSER
RKEKSRDAARSRRSKESEVFYELAHQLPLPHNVSSHLDKASVMRLTISYLRVRKLLDAGDLD
IEDDMKAQMNCFYLKALDGFVMVLTDDGDMIYISDNVNKYMGLTQFELTGHSVFDFTHPCDH
EEMREMLTHRNGLVKKGKEQNTQRSFFLRMKCTLTSRGRTMNIKSATWKVLHCTGHIHVYDT
NSNQPQCGYKKPPMTCLVLICEPIPHPSNIEIPLDSKTFLSRHSLDMKFSYCDERITELMGY
EPEELLGRSIYEYYHALDSDHLTKTHHDMFTKGQVTTGQYRMLAKRGGYVWVETQATVIYNT
KNSQPQCIVCVNYVVSGIIQHDLIFSLQQTECVLKPVESSDMKMTQLFTKVESEDTSSLEDK
LKKEPDDTDLDLEMLAPYIPMDDDFQLRSFDQLSPLESSSASPESASPQSTVTVFQQTQIQE
PTANATTTTATTDELKTVTKDRMEDIKILIASPSPTHIHKETTSATSSPYRDTQSRTASPNR
AGKGVIEQTEKSHPRSPNVLSVALSQRTTVPEEELNPKILALQNAQRKRKMEHDGSLFQAVG
IGTLLQQPDDHAATTSLSWKRVKGCKSSEQNGMEQKTIILIPSDLACRLLGQSMDESGLPQL
TSYDCEVAAPIQGSRNLLQGEELLRALDQVNEFEQKLISEEDL
mCherry Sequence (SEQ ID NO: 7):
MVSKGEEDNMAIIKEFMRFKVHMEGSVNGHEFEIEGEGEGRPYEGTQTAKLKVTKGGPLPFA
WDILSPQFMYGSKAYVKHPADIPDYLKLSFPEGFKWERVMNFEDGGVVTVTQDSSLQDGEFY
KVKLRGTNFPSDGPVMQKKTMGWEASSERMYPEDGALKGEIKQRLKLKDGGHYDAEVKTTYK
AKKPVQLPGAYNVNIKLDITSHNEDYTIVEQYERAEGRHSTGGMDELYK
GALA_CD81_Intein_scHIF1α Sequence (SEQ ID NO: 8):
MDYKDDDDKSRGVEGCTKCIKYLLFVFNFVFWLAGGVILGVALWLRHDPQTTNLLYLELGDK
PAPNTFYVGIYILIAVGAVMMFVGFLGCYGAIQESQCLLGTFFTCLVILFACEVAAGIWGFV
NKDQIAKDVKQFYDQALQQAVVDDGALADANNAKAVVKTFHETLDCCGSSTLTALTTSVLKN
NLCPSGSNIISNLFKEDCHQKIDDLFSGKLYLIGIAAIVVAVIMIFEMILSMVLCCGIRNSS
VYKLALAEGTRIFDPVTGTTHRIEDVVGGRKPIHVVAAAKDGTLHARPVVSWFDQGTRDVIG
LRIAGGAILWATPDHKVLTEYGWRAAGELRKGDRVAQPRREDGFGDSAPIPARVQALADALD
DKFLHDMLAEELRYSVIREVLPTRRARTFGLEVEELHTLVAEGVVVHNCMEGAGGANDKKKI
SSERRKEKSRDAARSRRSKESEVFYELAHQLPLPHNVSSHLDKASVMRLTISYLRVRKLLDA
GDLDIEDDMKAQMNCFYLKALDGFVMVLTDDGDMIYISDNVNKYMGLTQFELTGHSVFDFTH
PCDHEEMREMLTHRNGLVKKGKEQNTQRSFFLRMKCTLTSRGRTMNIKSATWKVLHCTGHIH
VYDTNSNQPQCGYKKPPMTCLVLICEPIPHPSNIEIPLDSKTFLSRHSLDMKFSYCDERITE
LMGYEPEELLGRSIYEYYHALDSDHLTKTHHDMFTKGQVTTGQYRMLAKRGGYVWVETQATV
IYNTKNSQPQCIVCVNYVVSGIIQHDLIFSLQQTECVLKPVESSDMKMTQLFTKVESEDTSS
LFDKLKKEPDDTDLDLEMLAPYIPMDDDFQLRSFDQLSPLESSSASPESASPQSTVTVFQQT
QIQEPTANATTTTATTDELKTVTKDRMEDIKILIASPSPTHIHKETTSATSSPYRDTQSRTA
SPNRAGKGVIEQTEKSHPRSPNVLSVALSQRTTVPEEELNPKILALQNAQRKRKMEHDGSLF
QAVGIGTLLQQPDDHAATTSLSWKRVKGCKSSEQNGMEQKTIILIPSDLACRLLGQSMDESG
LPQLTSYDCEVAAPIQGSRNLLQGEELLRALDQVNEFEQKLISEEDL
ESM_Intein_scHIF1α Sequence (SEQ ID NO: 9):
MDYKDDDDKSRKYPLLIGIGLSAVIGLLSCLIGYCSSKLALAEGTRIFDPVTGTTHRIEDVV
GGRKPIHVVAAAKDGTLHARPVVSWFDQGTRDVIGLRIAGGAILWATPDHKVLTEYGWRAAG
ELRKGDRVAQPRRFDGFGDSAPIPARVQALADALDDKFLHDMLAEELRYSVIREVLPTRRAR
TFGLEVEELHTLVAEGVVVHNCMEGAGGANDKKKISSERRKEKSRDAARSRRSKESEVFYEL
AHQLPLPHNVSSHLDKASVMRLTISYLRVRKLLDAGDLDIEDDMKAQMNCFYLKALDGFVMV
LTDDGDMIYISDNVNKYMGLTQFELTGHSVFDFTHPCDHEEMREMLTHRNGLVKKGKEQNTQ
RSFFLRMKCTLTSRGRIMNIKSATWKVLHCTGHIHVYDTNSNQPQCGYKKPPMTCLVLICEP
IPHPSNIEIPLDSKTFLSRHSLDMKFSYCDERITELMGYEPEELLGRSIYEYYHALDSDHLT
KTHHDMFTKGQVTTGQYRMLAKRGGYVWVETQATVIYNTKNSQPQCIVCVNYVVSGIIQHDL
IFSLQQTECVLKPVESSDMKMTQLFTKVESEDTSSLFDKLKKEPDDTDLDLEMLAPYIPMDD
DFQLRSFDQLSPLESSSASPESASPQSTVTVFQQTQIQEPTANATTTTATTDELKTVTKDRM
EDIKILIASPSPTHIHKETTSATSSPYRDTQSRTASPNRAGKGVIEQTEKSHPRSPNVLSVA
LSQRTTVPEEELNPKILALQNAQRKRKMEHDGSLFQAVGIGTLLQQPDDHAATTSLSWKRVK
GCKSSEQNGMEQKTIILIPSDLACRLLGQSMDESGLPQLTSYDCEVAAPIQGSRNLLQGEEL
LRALDQVNEFEQKLISEEDL
GALA_ESM_Intein_scHIF1α Sequence (SEQ ID NO: 10):
MDYKDDDDKSRGALAKYPLLIGIGLSAVIGLLSCLIGYCSSKLALAEGTRIFDPVTGTTHRI
EDVVGGRKPIHVVAAAKDGTLHARPVVSWFDQGTRDVIGLRIAGGAILWATPDHKVLTEYGW
RAAGELRKGDRVAQPRREDGFGDSAPIPARVQALADALDDKFLHDMLAEELRYSVIREVLPT
RRARTFGLEVEELHTLVAEGVVVHNCMEGAGGANDKKKISSERRKEKSRDAARSRRSKESEV
FYELAHQLPLPHNVSSHLDKASVMRLTISYLRVRKLLDAGDLDIEDDMKAQMNCFYLKALDG
FVMVLTDDGDMIYISDNVNKYMGLTQFELTGHSVFDFTHPCDHEEMREMLTHRNGLVKKGKE
QNTQRSFFLRMKCTLTSRGRTMNIKSATWKVLHCTGHIHVYDTNSNQPQCGYKKPPMTCLVL
ICEPIPHPSNIEIPLDSKTFLSRHSLDMKFSYCDERITELMGYEPEELLGRSIYEYYHALDS
DHLTKTHHDMFTKGQVTTGQYRMLAKRGGYVWVETQATVIYNTKNSQPQCIVCVNYVVSGII
QHDLIFSLQQTECVLKPVESSDMKMTQLFTKVESEDTSSLFDKLKKEPDDTDLDLEMLAPYI
PMDDDFQLRSFDQLSPLESSSASPESASPQSTVTVFQQTQIQEPTANATTTTATTDELKTVT
KDRMEDIKILIASPSPTHIHKETTSATSSPYRDTQSRTASPNRAGKGVIEQTEKSHPRSPNV
LSVALSQRTTVPEEELNPKILALQNAQRKRKMEHDGSLFQAVGIGTLLQQPDDHAATTSLSW
KRVKGCKSSEQNGMEQKTIILIPSDLACRLLGQSMDESGLPQLTSYDCEVAAPIQGSRNLLQ
GEELLRALDQVNEFEQKLISEEDL

The above-mentioned plasmids were amplified and isolated according to a protocol of the Qiagen® Plasmid Maxi kit. More specifically, 1 μl (0.1 ug) of the plasmid DNA and 100 μl competent cells DH5α were mixed in a 1.5 ml microcentrifuge tube. Plasmid DNA was introduced to competent cells DH5α by heat shock. To elaborate, the microcentrifuge tube containing the mixture of plasmid DNA and competent cells DH5α was heated at 42° C. for 45 seconds using a heat block. Following this, the heated microcentrifuge tube was placed on ice for 2 minutes. After cooling down, 90011.1 antibiotic-free room temperature LB agar media was added to the microcentrifuge tube. Then, this microcentrifuge tube was incubated at 37° C. for 45 minutes on a 200-rpm shaker. After incubation, 100 μl from the microcentrifuge tube was spread onto LB media containing plates with 100 μg/ml ampicillin. All plates were incubated overnight at 37° C. On the following day, a colony was taken from the surface of the plate and incubated in 2-3 ml of LB media with 100 μg/ml ampicillin at 37° C. for 8 hours. After incubation, 1 ml from the mixture of colony and LB media with antibiotics was transferred to a flask containing 500 ml of LB/ampicillin media and incubated overnight at 37° C. The bacterial cells were harvested by centrifugation at 6000×g for 15 min at 4° C., and the bacterial pellet was resuspended in Buffer P1 with RNase A 100 μg/ml. Buffer P2 was added and mixed thoroughly by vigorously inverting the sealed tube 4-6 times, and the resulting mixture was incubated at room temperature for 5 min. Chilled Buffer P3 was added and mixed immediately and thoroughly by vigorously inverting 4-6 times, and the resulting mixture was incubated on ice for 20 min. After centrifuging at ≥20,000×g for 30 min at 4° C., supernatant containing plasmid DNA was collected promptly. After centrifuging the supernatant again at ≥20,000×g for 15 min at 4° C., supernatant containing plasmid DNA was collected promptly. After equilibrating a QIAGEN-tip 500 by applying Buffer QBT and allowing the column to empty by gravity flow, the collected supernatant was applied to the QIAGEN-tip and allowed to enter the resin by gravity flow. After washing the QIAGEN-tip with Buffer QC, DNAs were eluted with Buffer QC. The eluted DNAs were precipitated by adding room-temperature isopropanol to the eluted DNA. After mixing and centrifuging immediately at ≥15,000×g for 30 min at 4° C., the supernatant was carefully decanted. After washing DNA pellet with room-temperature 70% ethanol and centrifuging at ≥15,000×g for 10 min, the supernatant was carefully decanted without disturbing the pellet. After air-drying the pellet for 5-10 min, and the final plasmid DNAs were redissolved in a suitable volume of buffer.

### Example 2

### Isolation of EVs

HEK293 cells (6×10⁶) were incubated at 37° C. with 5% CO₂ in Dulbecco's modified Eagle's medium (DMEM) to which 10% fetal bovine serum (FBS) was added. At the time when it had 8090% of confluency, the cells were transfected by transient transfection using transfecting reagents.

The cells were transfected by transient transfection using transfecting agents, lipofectamine 2000, lipofectamine 3000, or polyethylenimine (PEI). Cell medium was replaced with DMEM, and mixture of DNA and transfection reagent was added into the cells. The cells were then incubated at 37° C. with 5% CO₂ for 72 hours. To isolate EVs, the supernatants of cells were harvested when 72 hours were passed after the transfection. The supernatants were centrifuged at 300 g for 10 min, 2000 g for 10 min, and 10,000 g for 30 min. The supernatants were then filtered and concentrated with a tangential flow filtration (TFF) system. After that, the supernatants were centrifuged at 150,000 g for 1.5 hours. The EV pellets were resuspended with PBS (phosphate-buffered saline) including s proteinase inhibitor cocktail and preserved at 4° C. The protein concentration of the separated EVs was measured using a BCA protein analysis kit (Bio-Rad). See, e.g., Kim et al., Xenogenization of tumor cells by fusogenic exosomes in tumor microenvironment ignites and propagates antitumor immunity, Science Advances, 2020 Jul. 1;6(27): eaaz2083 which is incorporated herein by reference.

### Example 3

### Verification of Intein Function

Intein was used to load macromolecules into the EV Here, intein refers to a mutant peptide that only causes C-terminal cleavage, not ligation, from the existing recA mini-intein peptide of mycobacterium tuberculosis. The mini-Intein was removal of the central endonuclease domain of the recA Intein. The C2A, D25G, V68L and D423G of recA mini-intein were modified. Utilizing this mutated intein, the plasmid shown in FIG. 1 was created. To verify that scHIF1α is well encapsulated in the EV in a free-form by intein, a plasmid that does not get cut was created by mutating the Asn and Cys that are cut between intein of CD81_Intein and scHIF1α to Ala, and it was named CD81_Intein*_scHIFla (Mut) (SEQ ID NO: 6) plasmid (FIG. 2). As an additional control group, a plasmid was created that has a stop codon after intein in the CD81_Intein_scHIF1α plasmid, and it was named CD81_Intein (Stop) (SEQ ID NO: 5) plasmid (FIG. 2). The 3 plasmids, CD81_Intein_scHIF1α (Full) (SEQ ID NO: 4), CD81_Intein*_scHIF1α (Mut) (SEQ ID NO: 6), and CD81_Intein (Stop) (SEQ ID NO: 5), were transfected into HEK293T cells through PEI, and EVs were extracted.

Characteristic analysis of the EVs was performed via western blot. Western blot samples were prepared by adding 5x SDS 500mM TCEP and DW to make it 10 μg/10 μl. Western blot samples were incubated in a 95° C. heat block for 5 minutes. For the SDS gel, a gradient gel from Bio-Rad was used. The gel was run at 60 V for approximately an hour and a half, and the proteins on the SDS gel were transferred to an NC (nitrocellulose) membrane using Bio-Rad's trans-blot turbo transfer system. After creating 5% skim milk with a TBST (Tris-buffered saline with 0.05% Tween° 20 detergent) solution for 1-hour NC membrane blocking, myc, alix, tsg101, and calnexin antibodies were added to the 5% skim milk and left overnight at 4° C. The solution was washed four times for 10 minutes each with a TB ST solution. A rabbit secondary antibody (sigma) was added to 5% skim milk at a 1:3000 ratio and incubated at room temperature for 1 hour. Afterwards, it was washed four times for 10 minutes each with a TBST solution and then photographed using Bio-Rad's ECL (enhanced chemiluminescence) solution with a ChemiDoc imaging system at an exposure of 0.1 seconds.

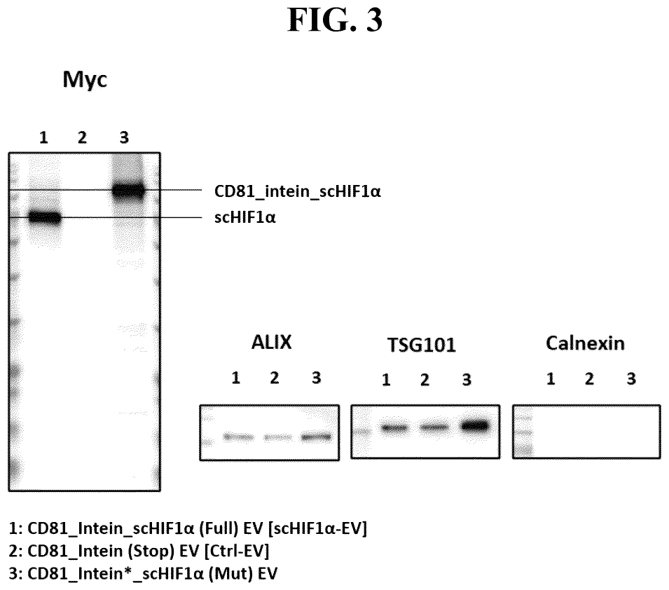

scHIF1α, which was not observed in CD81_Intein EVs [Ctrl-EVs], was observed in CD81_Intein scHIF1α (Full) EVs [scHIF1α-EVs] and CD81_Intein*_scHIF1α (Mut) EVs (myc antibody, the expression of scHIF1α is represented by the myc antibody), because the intein of CD81_Intein*_scHIF1α (Mut) in EVs was not cut, so scHIF1α was fused with the membrane while attached to intein, not in free-form. These results suggest that in the case of CD81_Intein_scHIF1α EV, the intein was properly cleaved as expected, implying that scHIF1α was well loaded into the EV. Furthermore, the observation of alix and tsg101, positive markers of EV, in all three EVs and the non-detection of the negative marker calnexin confirmed the successful separation of EVs (FIG. 3).

### Example 4

### Characterization of scHIF1α Packaging in EVs

To confirm the packaging efficiency of scHIF1α in CD81_Intein_scHIF1α (Full) EVs, EVs were extracted from HEK293T cells that had been transfected with the CD81_Intein_scHIF1α (Full) plasmid (SEQ ID NO: 4) and the pcDNA3.1-based scHIF1α (SEQ ID NO: 3) plasmid. Additionally, to mitigate variables associated with transfection efficiency, we utilized the pcDNA3.1-based mCherry (SEQ ID NO: 7) plasmid. For the cell lysates, RIPA (radioimmunoprecipitation) lysis buffer was used, with 3 rounds of vortexing at 10-minute intervals, followed by centrifugation at 13,000 rpm for 10 minutes. The protein concentrations of the cell lysates and EVs were measured using a BCA assay, and western blot samples were prepared by adding 5×SDS 500 mM TCEP and DW to make it 10 μg/10 μl. Western blotting was performed using the previously mentioned methods, utilizing myc and mCherry antibodies.

To account for potential discrepancies in transfection efficiency, cells were co-transfected with a pcDNA3.1-based mCherry (SEQ ID NO: 7) plasmid under identical conditions. Subsequent western blot analysis confirmed the expression of mCherry in both cells and EVs. The results demonstrated that mCherry expression was indistinguishable between cells transfected with the CD81_Intein_scHIF1α (Full) plasmid (SEQ ID NO: 4) or the pcDNA3.1-based scHIF1α (SEQ ID NO: 3) plasmid, as well as the derived EVs. The experimental results also indicated that the ratio of scHIF1α expression in the cell lysates was approximately 1:3 between the pcDNA3.1-based scHIF1α (SEQ ID NO: 3) plasmid group and the CD81_Intein_scHIF1α (Full) plasmid (SEQ ID NO: 4) group. Notably, in the EVs, this ratio increased to about 1:25. When using the CD81_Intein_scHIF1α (Full) plasmid (SEQ ID NO: 4), there was a more effective increase in scHIF1α expression within the EVs relative to the cell lysates (FIG. 4).

### Example 5

### Efficiency of scHIF1α Packaging in EVs

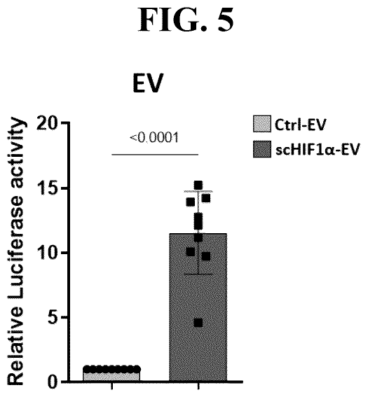

To verify that scHIF1α-EVs, were effectively delivering scHIF1α to targeted cells, we employed engineered HEK293T cells designed to express luciferase under the control of the VEGF promoter. These cells were treated with 30 μg of EVs per 3×10⁵ cells. Luminescence was subsequently assessed using the Promega Luciferase Assay System. The luminescence observed from CD81_Intein_scHIF1α (Full) EVs [scHIF1α-EV] was found to be markedly higher than that of both the PBS control and the CD81_Intein (Stop) EVs [Ctrl-EV] (FIG. 5).

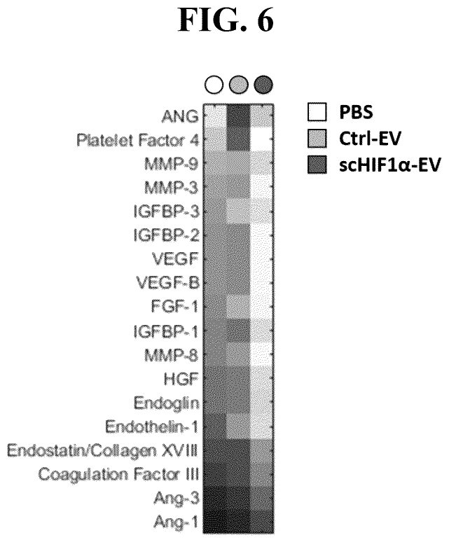

To confirm whether scHIF1α-EV actually induces the efficacy of HIF1α, we conducted an ex vivo cytokine array experiment related to angiogenesis. Macrophages were sorted from the liver tissue of C57BL/6 mice using F4/80 beads, and 3×10⁵ sorted macrophages were seeded in a 96 well round type plate. Each well was treated with 1 μg of scHIFα-EV and left in a 37° C. incubator for 24 hours. Afterwards, only the supernatant, with the cells removed, was used for the cytokine array analysis. The intensity corresponding to each cytokine was measured and compared between groups. The experimental results confirmed that compared to PBS or Ctrl-EV, scHIF1α-EV more effectively promoted the release of angiogenesis-related cytokines from macrophages (FIG. 6).

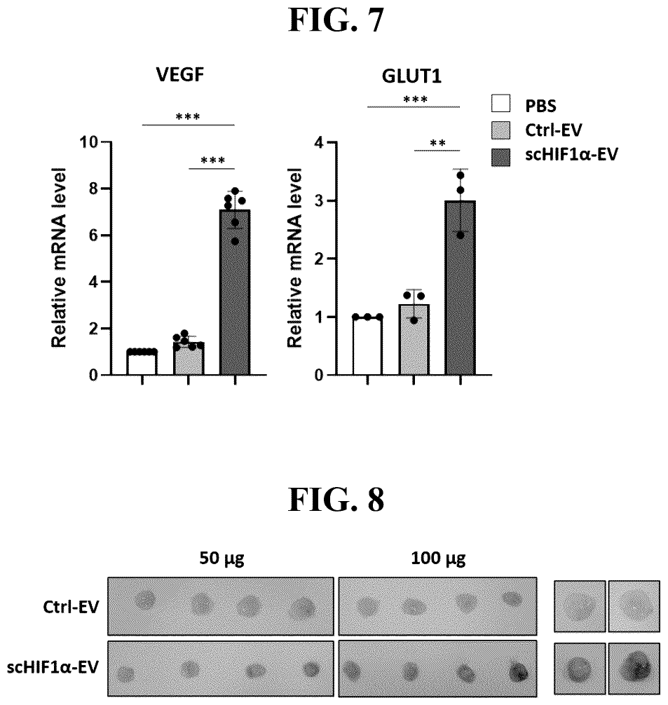

To verify the proper functioning of scHIF1α-EV in target cells, VEGF and GLUT1, known downstream signaling pathways of HIF1α, were confirmed through qRT-PCR. EVs were administered at 1 μg/ml to HEK293T cells, which were then cultured for 6 hours. Afterwards, all cells were collected, and RNA was extracted using the MN nucleospin RNA mini kit for RNA purification. Subsequently, cDNA was synthesized using the iScriptTM cDNA Synthesis Kit, and qPCR was performed using the SYBR™ Green PCR Master Mix. As a result, only the group treated with scHIF1α-EV showed a statistically significant increase in VEGF expression by approximately seven times, and GLUT1 expression also increased by about 3 times (FIG. 7).

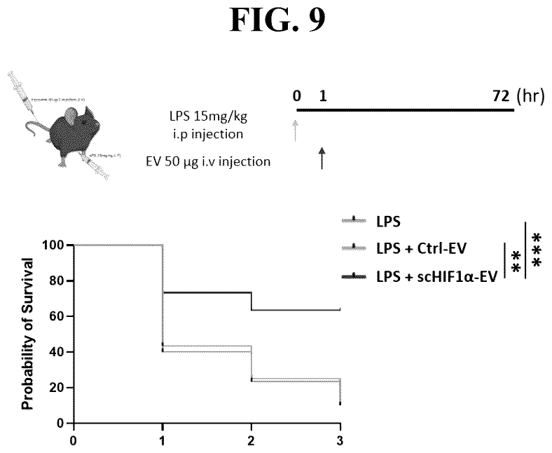

HIF1α is known as a master transcription factor involved in angiogenesis. To confirm the effect of angiogenesis, matrigel and extracellular vesicles (100 μg) were mixed in a volume ratio of 1:3, and a total of 500 μl was implanted into the flank of a NOD/SCID mouse. 2 weeks later, all implanted matrigel was removed to evaluate the degree of angiogenesis. Experimental results confirmed that the group treated with scHIF1α-EV stimulated angiogenesis within the matrigel more than the Ctrl-EV group (FIG. 8).

### Example 6

### Efficacy Verification of scHIF1α-EV in LPS-Induced Liver Injury Model

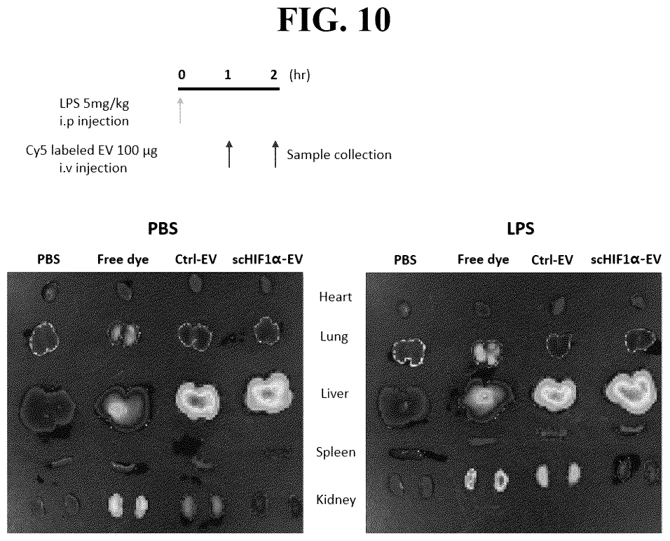

To verify the survival rate of scHIF1α-EV in a severe inflammatory disease model, a 7-week-old C57BL/6 male mouse was given an intraperitoneal injection of 15 mg/kg lipopolysaccharide (LPS). The intraperitoneal injection of LPS in mice induces severe inflammation and sepsis, leading to injury in various organs, including the liver. 1 hour later, 50 μg of EVs were administered via intravenous injection. Subsequently, monitoring was conducted for 3 days, confirming a statistically significant improvement in survival rate with a single injection of scHIF1α-EV (FIG. 9).

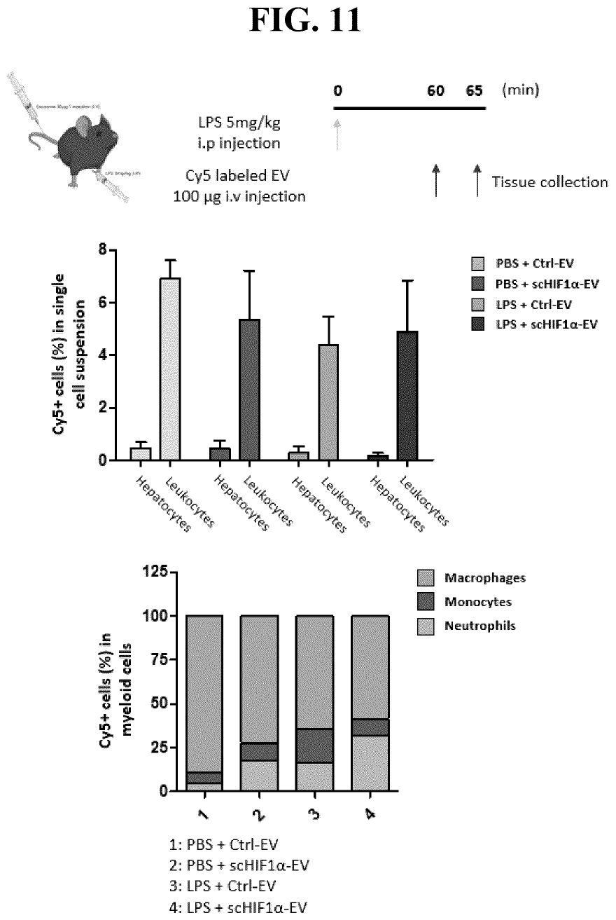

To evaluate the biodistribution of injected EVs in the body, EVs stained with Cy5 were administered intravenously, and tissues were excised 1 hour later for confirmation through an In vivo Imaging system (IVIS). EVs at a concentration of 1 mg/ml were stained 10 min with 100 μM Cy5, and free dye was removed using Zeba spin Desalting Columns 40K. Afterwards, the Cy5-stained EVs were adjusted for equal fluorescence values between groups. 1 hour later, the heart, lungs, liver, spleen, and kidneys were excised, and fluorescence values were evaluated through IVIS. Regardless of LPS treatment, it was observed that most EVs accumulated in the liver one hour later, regardless of the group (FIG. 10).

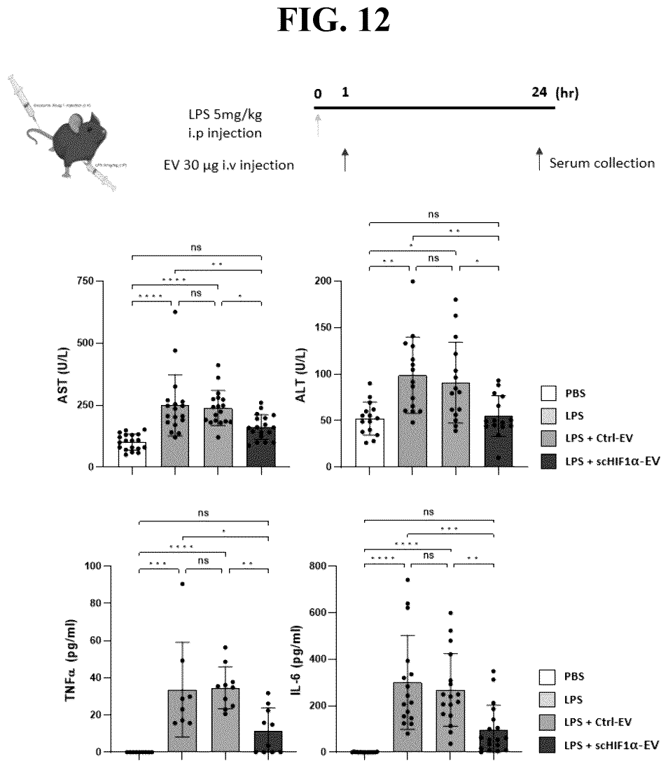

To analyze the cell types in which scHIF1α-EV accumulates within the inflamed liver tissues caused by an intraperitoneal injection of 5 mg/kg LPS, single cells were collected from the liver tissues 5 minutes after the intravenous injection of Cy5 labeled Ctrl -EV or scHIF1α-EV. The proportion of Cy5+ cells, which indicate the cells that have taken up the EVs, was determined by flow cytometry using specific antibodies (markers for each cell type: leukocytes as CD45+, hepatocytes as CD45−, macrophages as CD45+CD11b+F4/80+, monocytes as CD45+CD11b+F4/80^{low}Ly6C^{high}, and neutrophil as CD45+CD11b+Ly6G+). The results demonstrated that EVs preferentially targeted leukocytes, especially macrophages (>50%) (FIG. 11).

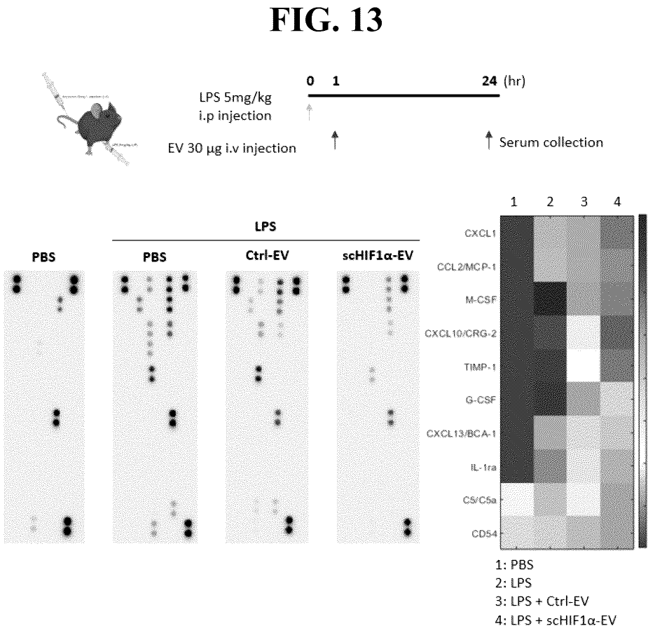

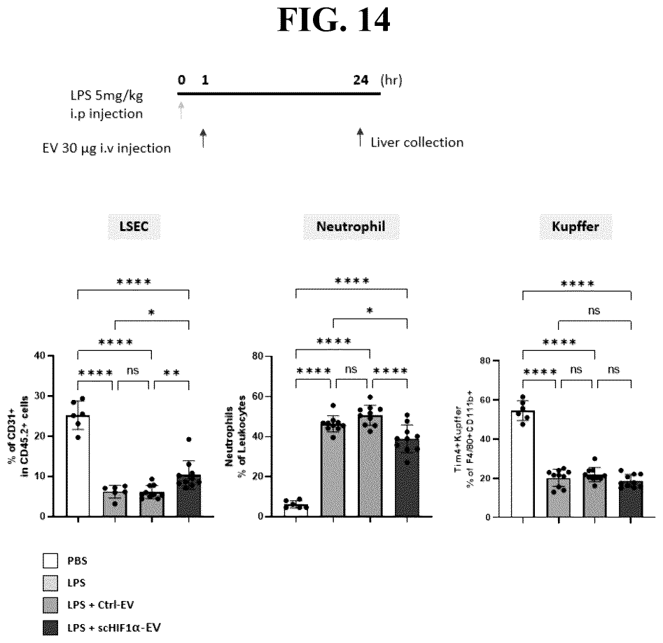

To verify the efficacy on LPS-induced liver injury, an intraperitoneal injection of 5 mg/kg LPS was administered, followed by an intravenous injection of 30 μg of scHIF1α-EV an hour later. After 24 hours post-induction, blood was drawn to measure liver toxicity-related ALT and AST, along with pro-inflammatory cytokines IL-6 and TNF-a in the serum. Statistically significant reductions in liver toxicity indices (AST, ALT) and pro-inflammatory cytokines (IL-6, TNF-α) were confirmed in the scHIF1α-EV group compared to the Ctrl-EV and untreated LPS control groups, as evidenced through cytokine ELISA (FIG. 12). Moreover, a cytokine array ELISA was conducted to analyze the overall cytokine profile in the serum separated from the blood. Compared to untreated group, a general decrease in pro-inflammatory cytokine and chemokines was observed in the scHIF1α-EV group (FIG. 13).

### Example 7

### Immune Cell Analysis in Liver Tissue in LPS-Induced Liver Injury Model

To analyze the immune efficacy of scHIF1α-EV in an LPS-induced liver injury model, LPS (5 mg/kg) was intraperitoneally injected into 7-week-old male C57BL/6 mice to induce a severe inflammatory condition. Subsequently, 30 μg of scHIF1α-EV was intravenously injected. After 24 hours, the immune cell analysis in damaged liver tissue was evaluated.

To assess the immune efficacy, liver tissues were harvested, treated with collagenase D, dispase, and RNase, and ground using a MACS dissociator. Samples obtained after filtering the debris through a 40 μm strainer were centrifuged at 50 g for 3 minutes. The supernatant was then centrifuged again at 300 g for 3 minutes to obtain the settled cells, which were then treated with 3 ml of RBC (red blood cell) lysis for 10 minutes at room temperature. The cells were then centrifuged again at 300 g for 3 minutes, the settled cells were washed with PBS, and then Fc blocker and antibody cocktail were attached for 40 minutes at 4° C. in a dark condition. Cells were then washed with PBS, and immune analysis was conducted through flow cytometry analysis. Markers for each cell were stained as follows: liver sinusoidal endothelial cells (LSEC) as CD45+CD11b-CD31+, neutrophils as CD45+CD11b+Ly6G+, monocytes as CD45+CD64−CD11b+F4/80^{low}, monocyte-derived macrophages (MoMF) as CD45+CD64+CD11b+F4/80+, and kupffer cells as CD45+CD64+CD11b+F4/80+Tim4+.

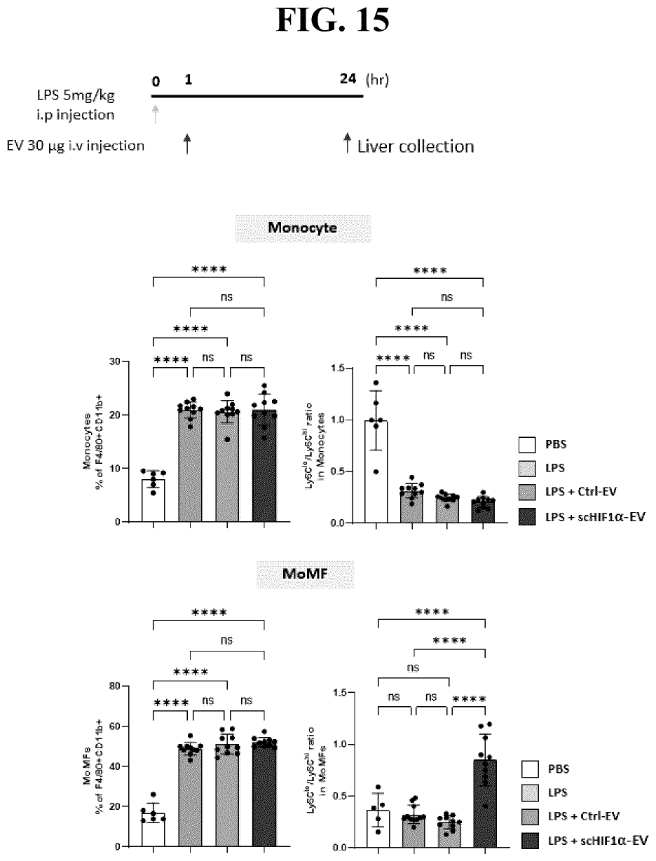

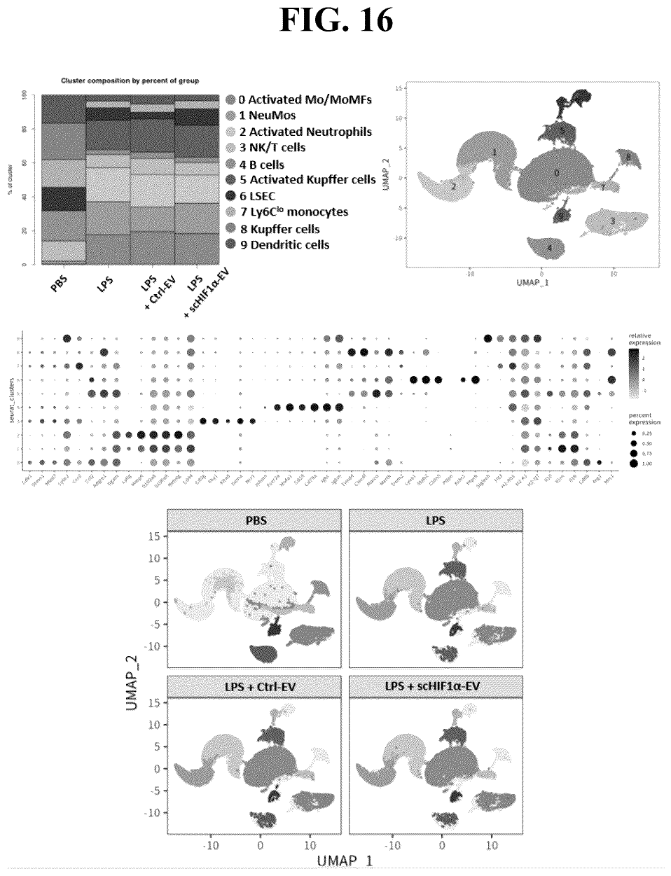

The immunological analysis revealed that there was a statistically significant increase in the proportion of liver sinusoidal endothelial cells (LSEC) CD45+CD131+ and a decrease in the proportion of neutrophils, which are related to acute inflammatory responses, in the scHIF1α-EV treatment group (FIG. 14). Also, while there was no statistically significant difference in the ratio of kupffer cells, monocytes and MoMFs between different control groups, the ratio of MoMFs expressing the immune cell type Ly6C at a low level, which reduces inflammation and promotes regeneration, increased with scHIF1α-EV treatment. As a result, there was a significant increase in the ratio of Ly6C^{lo} (low)/Ly6C^{hi} (high) within MoMFs in the scHIFla-EV treatment group compared to other control groups (FIG. 15).

### Example 8

### Elucidating the Mechanism of scHIF1α-EV through Single Cell RNA Sequencing

To elucidate the therapeutic mechanism of scHIF1α-EV in an LPS-induced liver injury model via single cell RNA sequencing, LPS (5 mg/kg) was intraperitoneally administered to 7-week-old male C57BL/6 mice, thereby inducing a severe inflammatory condition. Subsequently, 30 μg of scHIF1α-EV was administered intravenously. After 24 hours, single cell RNA sequencing was carried out. Liver tissues were harvested, subjected to treatment with collagenase D, dispase, and RNase, and subsequently homogenized using a MACS dissociator. Samples, after filtration of debris through a 40 μm strainer, were centrifuged at 50 g for 3 minutes. The supernatant was then further centrifuged at 300 g for 3 minutes to collect the pelleted cells. These cells were treated with 3 ml of RBC lysis buffer for 10 minutes at 4° C. After an additional centrifugation at 300 g for 3 minutes, the pelleted cells were washed with PBS and treated with CD45+ magnetic beads (Miltenyi Biotec) for the sorting of immune cells. Immune cells were sorted from single-cell suspensions according to the manufacturer's instructions. Following a final wash with PBS, the sorted immune cells were prepared for single cell RNA sequencing analysis using the 10× Genomics Chromium platform.

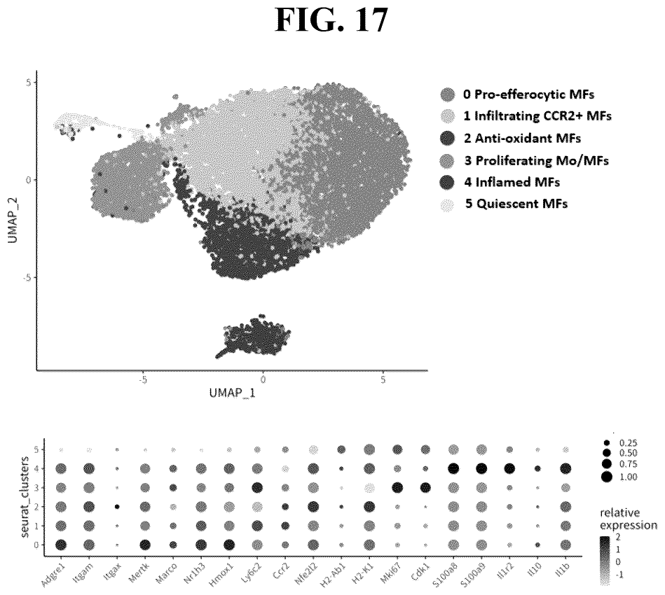

Through the analysis of the acquired single cell RNA, we identified 10 cell clusters: activated monocytes/monocyte derived macrophages (activated Mo/MoMFs, cluster 0), neutrophil-like monocytes (NeuMos, cluster 1), activated neutrophils (cluster 2), NK/T cells (cluster 3), B cells (cluster 4), activated kupffer cells (cluster 5), LSECs (cluster 6), Ly6C^{lo} monocytes (cluster 7), kupffer cells (cluster 8), and dendritic cells (cluster 9). Experimentally, it was observed that in a liver with induced severe inflammation, the proportion of activated monocytes/monocyte-derived macrophages (cluster 0) significantly increased compared to a normal liver (FIG. 16).

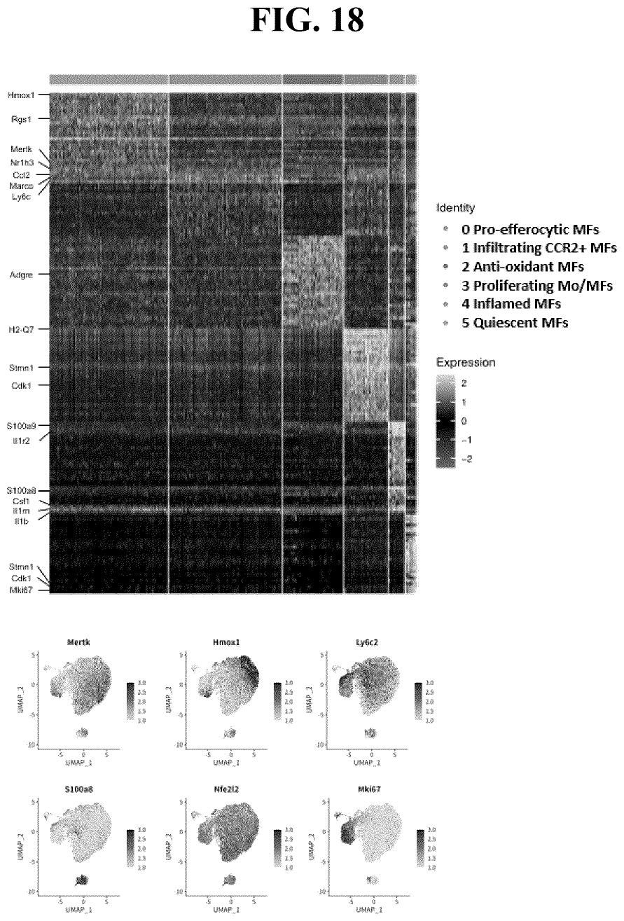

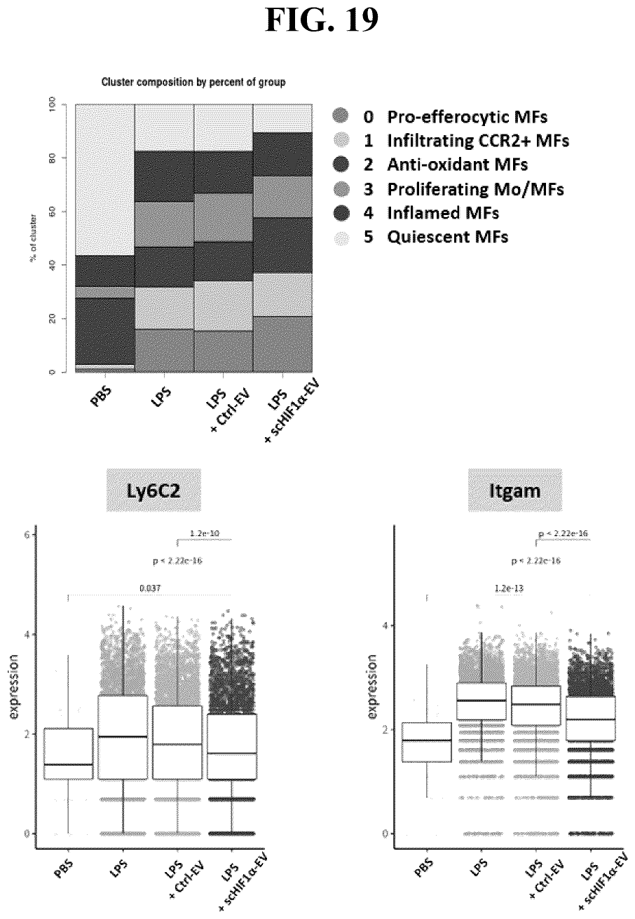

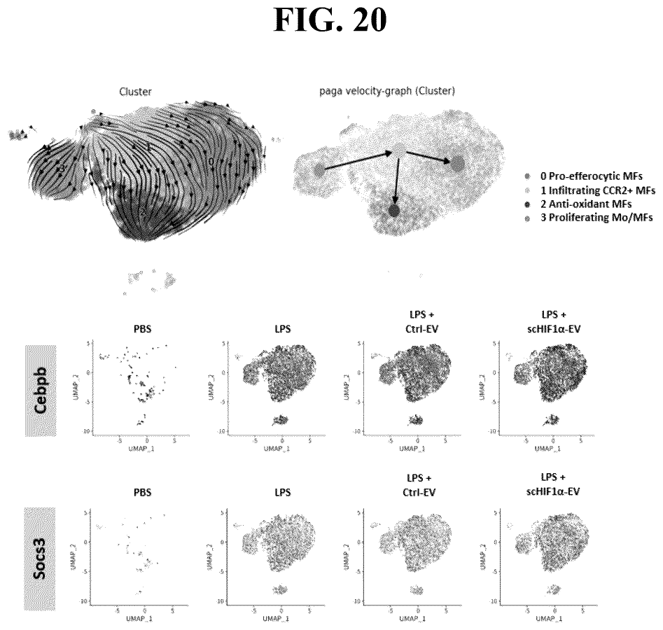

Cluster 0 in FIG. 16, composed of activated monocytes/monocyte derived macrophages, was further divided into 6 sub-clusters based on single cell RNA data: pro-efferocytic macrophages (pro-efferocytic MFs, cluster 0), infiltrating CCR2+ macrophages (infiltrating CCR2+ MFs, cluster 1), anti-oxidant macrophages (anti-oxidant MFs, cluster 2), proliferating monocytes/macrophages (proliferating Mo/MFs, cluster 3), inflamed macrophages (inflamed MFs, cluster 4), and quiescent macrophages (quiescent 1V1Fs, cluster 5) (FIGS. 17, 18). Experimental results revealed that treatment with scHIF1α-EV increased the proportions of pro-efferocytic macrophages (cluster 0) and anti-oxidant macrophages (cluster 2), both of which have the potential to promote anti-inflammatory effects and regeneration (FIG. 19). Furthermore, scHIF1α-EV significantly reduced the mRNA levels of Ly6C2 (which encodes Ly6C) and Itgam (which encodes CD11b) in the activated Mo/MoMFs cluster, suggesting an activation of resolutive macrophages (FIG. 19).

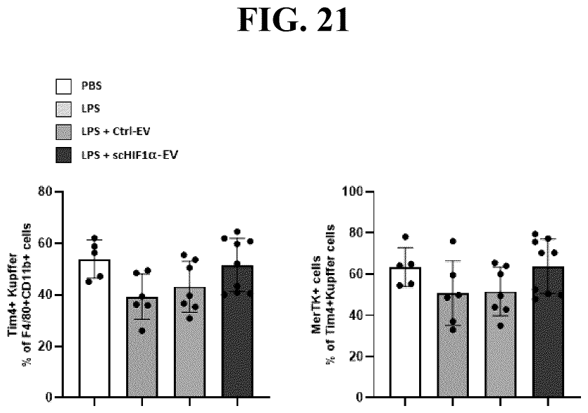

We determined the differentiation direction through trajectory analysis of the sub -clusters. We observed that clusters 0 and 2, whose proportions had increased in the scHIF1α-EV group, were highly differentiated macrophages. We also noted an increase in the expression of Cebpb, which is associated with anti-inflammatory myeloid differentiation, and Socs3, which inhibits pro-inflammatory cytokine signaling, in the scHIF1α-EV treated group (FIG. 20).

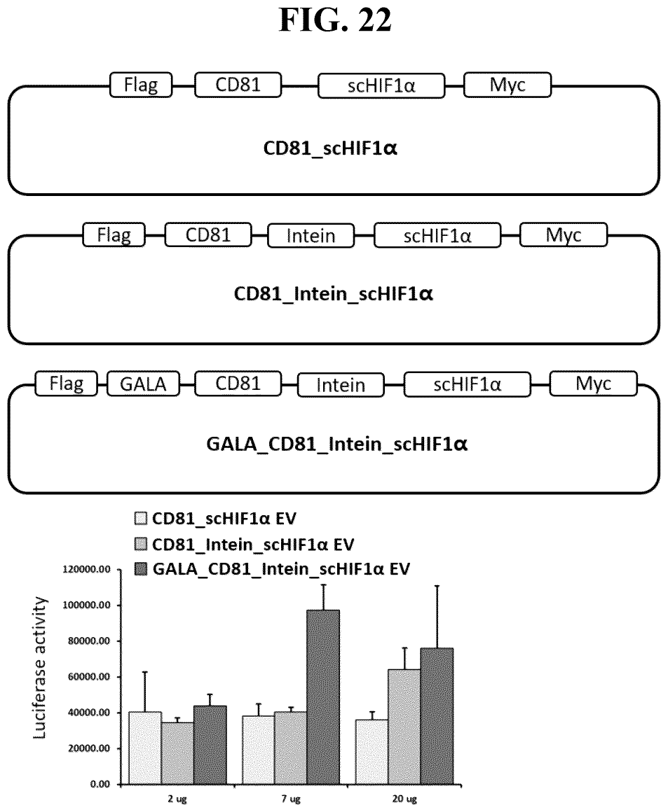

To evaluate whether the population of liver kupffer cells had normalized after 14 days, we collected liver tissue samples from mice that had received an intraperitoneal injection of LPS at a dose of 5 mg/kg, followed by an intravenous injection of 30 μg EVs an hour later. Our experimental data showed that the treatment with scHIF1α-EV resulted in an increase in the proportions of kupffer cells, including those that were MerTK-positive (FIG. 21). MerTK-positive macrophages, also known as efferocytic macrophages, have the capacity to enhance the resolution of inflammation within the liver, as described in Triantafyllou et al.. MerTK expressing hepatic macrophages promote the resolution of inflammation in acute liver failure, Gut. 2018 February; 67(2):333-347, which is incorporated herein by reference. The findings from this data suggest that treatment with scHIF1α-EV can induce phenotype switching in tissue macrophages toward a resolutive type capable of facilitating liver injury regeneration.

### Example 9

### Enhancement of scHIF1α Delivery Efficiency in EVs with Addition of GALA

To enhance the delivery efficiency of scHIF1α, we aimed to express the GALA peptide on the outer surface of the EV. Known for its ability to induce strong fusion with target cell membranes under acidic pH conditions, the GALA peptide consists of glutamic acid-alanine-leucine-alanine. The capability of the GALA peptide to fuse under acidic conditions can provide endosomal escape characteristics to EVs, thereby improving the intracellular delivery efficiency of scHIF1α. Consequently, the EVs produced by the GALA_CD81_Intein scHIF1α construct (SEQ ID NO: 8) were able to significantly enhance delivery efficiency in comparison to the existing EVs generated by the CD81-Intein-scHIF1α construct.

To evaluate the improved efficacy due to the increased delivery efficiency of scHIF1α into target cells with the addition of the GALA sequence, 1.5×10⁶ 293T cells were seeded in a 6-well plate and transfected with 200 ng of a plasmid with a luciferase tag attached to the VEGF promoter. 1 hour after transfection, 2, 7, and 20 pg of EVs obtained using the CD81_Intein, CD81_Intein_scHIF1α, GALA_CD81_Intein_scHIF1α plasmids were treated for 30 minutes in a 37° C. incubator. The cells were then replaced with DMEM high glucose media (10% FBS+1% antibiotic-antimycotic) and incubated under the same conditions for 18 hours. After washing with PBS, luciferase activity was measured to compare the level of VEGF activation caused by scHIF1α. Experimental results showed a higher luminescence from EVs produced using the GALA_CD81_Intein_scHIF1α plasmid compared to CD81_Intein_scHIF1α (SEQ ID NO: 4), suggesting that the incorporation of GALA leads to more efficient delivery of HIFla loaded in the EV (FIG. 22).

To maximize the efficiency of the existing construct, we fabricated the ESM-Intein-scHIF1α construct. The term ESM refers to an extracellular vesicle sorting motif. By using the construct, we were able to maximize the expression efficiency of the protein that we wanted to display on the EVs. The sequence of the ESM and the method of making the sequence are described in U.S. application Ser. No. 18/322,723, which is incorporated herein by reference.

In addition to increasing the efficiency of scHIF1α loading onto EVs, we aimed to maximize the delivery efficiency of scHIF1α by displaying the GALA peptide on the EV surface. To verify the efficacy of the improved construct, we treated HEK293T cells with EVs produced based on each construct and validated whether cell growth increased due to the delivered HIF1α via a CCK assay. Specifically, we seeded 4000 HEK293T cells per well in a 96-well plate, using DMEM high glucose (10% FBS+1% antibiotic-antimycotic) as the medium. After one day, the medium was suctioned off, and 10 μg of EVs, extracted from HEK293T cells transfected with CD81_intein_scHIF1α (SEQ ID NO: 4), ESM_Intein_scHIF1α (SEQ ID NO: 9), and GALA_ESM_Intein_scHIF1α (SEQ ID NO: 10), were treated with 200 μl of medium per well. After 24 hours, we added 10 μl of CCK solution per well. After 4 hours, we proceeded with imaging under a 480 nm condition with a microplate reader to assess the degree of cell proliferation. The results of the CCK assay confirmed that the EVs based on the GALA-ESM-Intein-scHIFla construct most significantly increased the viability of HEK293T cells (FIG. 23).

1. A method for inducing resolutive macrophage in a subject, the method comprising administering to the subject a therapeutically effective amount of one or more compositions that comprise one or more extracellular vesicles (EVs) and one or more pharmaceutically acceptable carriers and/or excipients, wherein the EV or EVs comprise one or more polypeptide constructs comprising: (i) HIF-1α, a fragment of the HIF-1α, a variant of the HIF-1α, a fragment of the variant, or a variant of the fragment; (ii) one or more EV polypeptides; and (iii) one or more monomeric cis-cleaving inteins.

2. The method of claim 1, wherein the composition modifies polarization of macrophages to favor resolutive macrophages and/or modifies balance between the different subtypes of macrophages toward resolutive macrophages and/or induces differentiation of monocytes to resolutive macrophages and/or induces phenotype switching from macrophages to resolutive macrophages.

3. The method of claim 1, wherein the fragment of the HIF-1α defects oxygen-dependent degradation domain (ODD) from wild-type HIF-1α.

4. The method of claim 1, wherein the variant of the HIF-1α comprises the amino acid sequence of SEQ ID NO: 3.

5. The method of claim 1, wherein the EV polypeptide or polypeptides are selected from the group consisting of:
one or more polypeptides selected from the group consisting of CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), and PTGFRN;
a fragment or fragments of the polypeptide or polypeptides;
a variant or variants of the polypeptide or polypeptides;
a variant or variants of the fragment or fragments; and
a fragment or fragments of the variant or variants.

6. The method of claim 1, wherein the monomeric cis-cleaving intein originates from the protein selected from the group consisting of Prp8, VMA1, DdRP, ThrRS, GLT, CHS, IF2 elF5B, DnaB, ClpP, RIR, Helicases, and MutS-like.

7. The method of claim 1, wherein the polypeptide construct or constructs further comprise one or more targeting moieties.

8. The method of claim 7, wherein the targeting moiety or moieties target one or more monocytes and/or one or more monocyte-derived macrophages.

9. The method of claim 1, wherein the polypeptide construct or constructs further comprise one or more fusogenic peptides.

10. The method of claim 9, wherein the fusogenic peptide or peptides are selected from the group consisting of (GALA)n (n is integer from 1 to 10), (KALA)n (n is integer from 1 to 10), INF7, influenza virus HA2, melittin, octa-arginine (R8) peptide, vesicular stomatitis virus glycoprotein (VSV-G), tat protein of HIV, HSV-1 gB, EBV gB, thgoto virus G protein, and AcMNPV gp64.

11. The method of claim 1, wherein the monomeric cis-cleaving intein comprises the amino acid sequence of SEQ ID NO: 2.

12. The method of claim 1, wherein the polypeptide construct or constructs comprise an amino acid sequence or sequences selected from the group consisting of SEQ ID NOs: 4, 8, 9 and 10.

13. The method of claim 1, wherein the subject is in status of (a) increased M1 and/or M1-like macrophages compared to normal condition and/or (b) inflammatory condition accompanying increased proportion and/or increased number of monocyte-derived macrophage compared to normal condition.

14. The method of claim 1, wherein the resolutive macrophage is selected from the group consisting of an Ly6C(low) macrophage and a MerTK-positive macrophage.

15. The method of claim 1, wherein the resolutive macrophage is selected from the group consisting of a pro-efferocytic macrophage and an anti-oxidant macrophage.

16. A resolutive macrophage-inducing agent comprising an EV or EVs that comprise one or more polypeptide constructs, wherein the polypeptide construct or constructs comprise: (i) HIF-1α, a fragment of the HIF-1α, a variant of the HIF-1α, a fragment of the variant, or a variant of the fragment; (ii) an EV polypeptide; and (iii) a monomeric cis-cleaving intein.

17. A method for preventing or treating inflammatory diseases, conditions, or symptoms, the method comprising administering to a subject a prophylactically or therapeutically effective amount of a composition containing one or more EVs that comprise one or more polypeptide constructs, wherein the polypeptide construct or constructs comprise: (i) HIF-1α, a fragment of the HIF-1α, a variant of the HIF-1α, a fragment of the variant, or a variant of the fragment; (ii) an EV polypeptide; and (iii) a monomeric cis-cleaving intein.

18. The method of claim 17, the inflammatory disease, condition, or symptom is related to (a) increased M1 and/or M1 -like macrophages compared to normal condition and/or (b) increased proportion and/or increased number of monocyte-derived macrophage compared to normal condition.

19. The method of claim 17, wherein the inflammatory disease, condition, or symptom is selected from the group consisting of single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, 15 tendinopathies, allergy, asthma, rheumatoid arthritis, glomerulonephritis, pancreatitis, hepatitis, non-alcoholic steatohepatitis (NASH), inflammatory arthritis, gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, crohn's and ulcerative colitis, terminal ileitis, alcoholic steatohepatitis, hepatotoxicity, liver infection, remote liver injury, lupus, autoimmune diseases associated with acute or chronic inflammation, and acute or chronic inflammation associated with viral, bacterial, or fungal infection.

20. An EV comprising one or more polypeptide construct or constructs that comprise (i) one or more fusogenic peptides, (ii) one or more polypeptides of interest (PoIs), (iii) one or more EV polypeptides, and (iv) one or more monomeric cis-cleaving inteins.
